(19) United States
(12) Patent Application Publication
(10) Pub. No.: US 2022/0296193 A1
(43) Pub. Date: Sep. 22, 2022

(54) FAST AND AUTOMATIC POSE ESTIMATION USING INTRAOPERATIVELY LOCATED FIDUCIALS AND SINGLE-VIEW FLUOROSCOPY

(71) Applicant: The Johns Hopkins Uniersity, Baltimore, MD (US)

(72) Inventors: Robert Grupp, Baltimore, MD (US); Russell H. Taylore, Severna Park, MD (US); Mehran Armand, Filton, MD (US)

(73) Assignee: The Johns Hopkins Uniersity, Baltimore, MD (US)

(21) Appl. No.: 17/639,546

(22) PCT Filed: Sep. 4, 2020
(86) PCT No.: PCT/US2020/049550

§ 371 Date: Mar. 1, 2022

Related U.S. Application Data
(60) Provisional application No. 62/896,271, filed on Sep. 5, 2019.

(51) Int. Cl. A61B 6/00; A61B 90/00

(52) U.S. Cl. CPC A61B 6/505; A61B 6/5235; A61B 90/37; A61B 90/39; A61B 2034/105

(57) ABSTRACT

A method of representing a relative change in position and/or orientation of a bone portion during a surgical procedure according to some embodiments of the invention includes receiving preoperative x-ray computed tomography (CT) images of a bone that will have a portion separated and moved during the surgical procedure. Multiple x-ray images are received, each a different view of the bone during the surgical procedure prior to separating and moving the portion, where the bone has a fiducial marker fixed relative to that portion and another fiducial marker fixed relative to a stationary portion of the bone. The fiducial markers each have at least three radio-opaque points and remain substantially fixed with respect to each other. The position of the radio-opaque points is determined relative to a three-dimensional representation of the bone from the preoperative CT images. After separating and moving the portion, a single x-ray image is received that includes both fiducial markers. The relative change in position and/or orientation is estimated using the single x-ray image.

### CROSS-REFERENCE OF RELATED APPLICATION

This application claims priority to U.S. Provisional Application No. 62/896,271, filed Sep. 5, 2019, the entire contents of which are hereby incorporated by reference.

This invention was made with government support under grant R01EB006839 and R21EB020113 awarded by the National Institutes of Health. The government has certain rights in the invention.

### BACKGROUND

### 1. Technical Field

Currently claimed embodiments of this invention relate to computerized systems and methods for use during surgical procedures, and more particularly computerized systems and methods representing relative changes in position and/or orientation of bone sections for use during surgical procedures.

### 2. Discussion of Related Art

Patients suffering from developmental dysplasia of the hip (DDH) typically have severe pain and reduced coverage of the femoral head, which can lead to joint osteoarthritis and subluxation of the femur [1]. Joint-preserving pelvic osteotomies, such as the Periacetabular osteotomy (PAO), treat DDH by reorienting the hip joint for increased femoral head coverage [2]. Specifically, for PAO, four osteotomies are performed about the acetabulum, fracturing it from the pelvis and allowing it to be adjusted to the desired pose [2]. In the conventional approach, PAO surgeons rely on 2D X-ray images, tactile feedback, experience and acumen to navigate the surgery [2]. Clinicians typically assess femoral head coverage intraoperatively using specific radiographic measurements, such as the lateral center edge (LCE) angle [3], derived from fluoroscopy [4]. However, this approach does not indicate the full 3D alignment of the acetabular fragment, nor does it describe additional biomechanical parameters, which have the potential to improve surgical outcomes [5-9]. A 3D example of a relocated fragment and a corresponding 2D fluoroscopic view is shown in FIG. 1.

Accordingly, there remains a need for improved computerized systems and methods for representing relative change in position and/or orientation of bone sections for use during surgical procedures.

### SUMMARY

A computerized system for representing a relative change in position and/or orientation of a bone section for use during a surgical procedure according to some embodiments of the invention includes data processing circuits configured to receive preoperative x-ray computed tomography (CT) image data of a bone that will have a portion separated and moved during the surgical procedure, and receive x-ray image data for multiple two-dimensional x-ray images, each two-dimensional x-ray image being a different view of the bone during the surgical procedure prior to having the portion separated and moved during the surgical procedure. The bone has a first fiducial marker fixed relative to said portion of the bone that will be separated and moved and a second fiducial marker fixed relative to a portion of the bone that will remain substantially stationary during the surgical procedure, the first and second fiducial markers each having at least three radio-opaque points to be identifiable in the multiple two-dimensional x-ray images and that remain substantially fixed with respect to each other within each respective first and second fiducial marker during the surgical procedure. The data processing circuits determine a position of the at least three radio-opaque points in each of the first and second fiducial markers relative to a three-dimensional representation of the bone from the preoperative CT image data of the bone, and receive after the portion of said bone is separated and moved during the surgical procedure, a single two-dimensional x-ray image data of at least a portion of the bone that includes both the first and second fiducial markers. The circuits estimate at least one of the relative change in position or the relative change of orientation of the portion of the bone that was separated and moved during the surgical procedure using the single two-dimensional x-ray image data, and provide information to a user representing at least one of the relative change in position or the relative change of orientation of the portion of the bone that was separated and moved during the surgical procedure based on the estimating.

A method for representing a relative change in position and/or orientation of a bone section for use during a surgical procedure according to some embodiments of the invention includes receiving preoperative x-ray computed tomography (CT) image data of a bone that will have a portion separated and moved during the surgical procedure, and receiving x-ray image data for multiple two-dimensional x-ray images, each two-dimensional x-ray image being a different view of the bone during the surgical procedure prior to having the portion separated and moved during the surgical procedure. The bone has a first fiducial marker fixed relative to said portion of the bone that will be separated and moved and a second fiducial marker fixed relative to a portion of the bone that will remain substantially stationary during the surgical procedure, the first and second fiducial markers each having at least three radio-opaque points to be identifiable in the multiple two-dimensional x-ray images and that remain substantially fixed with respect to each other within each respective first and second fiducial marker during the surgical procedure. The method determines a position of the at least three radio-opaque points in each of the first and second fiducial markers relative to a three-dimensional representation of the bone from the preoperative CT image data of the bone, and receives after the portion of said bone is separated and moved during the surgical procedure, a single two-dimensional x-ray image data of at least a portion of the bone that includes both the first and second fiducial markers. The method estimates at least one of the relative change in position or the relative change of orientation of the portion of the bone that was separated and moved during the surgical procedure using the single two-dimensional x-ray image data, and provides information to a user representing at least one of the relative change in position or the relative change of orientation of the portion of the bone that was separated and moved during the surgical procedure based on the estimating.

A computer-readable medium including computer-executable code for representing a relative change in position and/or orientation of a bone section for use during a surgical procedure according to some embodiments of the invention, which when executed by a computer causes the computer to receive preoperative x-ray computed tomography (CT) image data of a bone that will have a portion separated and moved during the surgical procedure, and receive x-ray image data for multiple two-dimensional x-ray images, each two-dimensional x-ray image being a different view of the bone during the surgical procedure prior to having the portion separated and moved during the surgical procedure. The bone has a first fiducial marker fixed relative to said portion of the bone that will be separated and moved and a second fiducial marker fixed relative to a portion of the bone that will remain substantially stationary during the surgical procedure, the first and second fiducial markers each having at least three radio-opaque points to be identifiable in the multiple two-dimensional x-ray images and that remain substantially fixed with respect to each other within each respective first and second fiducial marker during the surgical procedure. The computer is further caused to determine a position of the at least three radio-opaque points in each of the first and second fiducial markers relative to a three-dimensional representation of the bone from the preoperative CT image data of the bone, and receive after the portion of said bone is separated and moved during the surgical procedure, a single two-dimensional x-ray image data of at least a portion of the bone that includes both the first and second fiducial markers. The computer is further caused to estimate at least one of the relative change in position or the relative change of orientation of the portion of the bone that was separated and moved during the surgical procedure using the single two-dimensional x-ray image data, and provide information to a user representing at least one of the relative change in position or the relative change of orientation of the portion of the bone that was separated and moved during the surgical procedure based on the estimating.

### BRIEF DESCRIPTION OF THE DRAWINGS

FIG. 1 illustrates examples of an adjusted acetabular fragment visualized in 3D (a) and in a corresponding 2D fluoroscopic image (b). The fragment pose shown in (a) was estimated using the view shown in (b).

FIG. 2 summarizes the proposed surgical workflow for some embodiments, including the data required for each step.

FIG. 3 illustrates a bead injection device used in some embodiments to implant BB constellations after performing soft-tissue dissection, but prior to osteotomy.

FIG. 4 illustrates a visual example of the pre-osteotomy reconstruction process for a single BB in some embodiments.

FIG. 5 illustrates a workflow overview of the intraoperative BB reconstruction process of some embodiments.

FIG. 6 illustrates a complete workflow used in some embodiments for single-view relative pose estimation of the acetabular fragment.

FIG. 7 illustrates the data flow of the general pruning strategy used during BB constellation pose estimations in some embodiments.

FIG. 8 illustrates an overview of the ilium BB constellation pose estimation process in some embodiments.

FIG. 9 illustrates an example, on the top row, a pose pruned for excessive difference from the reference AP orientation. The bottom row corresponds to an example of a pose pruned due to a large mean fragment BB constellation re-projection distance.

FIG. 10 illustrates several examples of poses and correspondences used for initialization of the full-pelvis intensity-based, 2D/3D, registration.

FIG. 11 illustrates an example of an implausible fragment pose which was pruned due to a large rotation.

FIG. 12 illustrates a workflow of the fragment BB constellation pose estimation process.

FIG. 13 illustrates a toy example of the P3P problem showing the four possible solutions when mapping the BB constellation into the C-Arm coordinate frame.

FIG. 14 illustrates fluoroscopic images used for pose estimation in the cadaver surgeries.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Accurate and consistent mental interpretation of fluoroscopy to determine the position and orientation of acetabular bone fragments in 3D space is difficult. Accordingly, an embodiment of the current invention provides a computer assisted approach that uses a single fluoroscopic view and quickly reports the pose of an acetabular fragment without any user input or initialization.

In the following detailed description, the pelvis will be referred to in particular. However, the general concepts of the current invention can be applied to surgical procedures on other bones and/or combinations of bones, groups of three or more bones, as well as surgical tools and foreign implants, as will be discussed below.

Intraoperatively, but prior to any osteotomies, in some embodiments two constellations of metallic ball-bearings (BBs) are injected into the wing of a patient's ilium and lateral superior pubic ramus in one example according to an embodiment of the current invention. Although constellations of BBs will be used as examples in the following, the general concepts of the current invention are not limited to only using constellations of BBS. More generally, the constellations of BBs are examples of fiducial markers, each having at least three radio-opaque identifiable regions or points. In this example, one constellation is located on the expected acetabular fragment, and the other is located on the remaining, larger, pelvis fragment. The 3D locations of each BB are reconstructed using at least three fluoroscopic views and 2D/3D registrations to a preoperative CT scan of the pelvis. The relative pose of the fragment is established by estimating the movement of the two BB constellations using a single fluoroscopic view taken after osteotomy and fragment relocation. BB detection and inter-view correspondences are automatically computed throughout the processing pipeline.

An embodiment of the current invention was evaluated on a multitude of fluoroscopic images collected from six cadaveric surgeries performed bilaterally on three specimens. Mean fragment rotation error was 2.4±1.0 degrees, mean translation error was 2.1±0.6 mm, and mean 3D lateral center edge angle error was 1.0±0.5 degrees. The average runtime of the single-view pose estimation was 0.7±0.2 seconds. However, the general concepts of the current invention are not limited to these particular examples which are described in more detail in the Examples section below.

Examples according to some embodiments of the current invention demonstrate accuracy similar to other state of the art systems which require optical tracking systems or multiple-view 2D/3D registrations with manual input. The errors reported on fragment poses and lateral center edge angles are within the margins required for accurate intraoperative evaluation of femoral head coverage.

A processing pipeline according to an embodiment of the current invention is capable of automatically reporting fragment poses from a single fluoroscopic view with mean runtimes below one second. This pipeline is inspired by Roentgen stereometric analysis (RSA) techniques, which use metallic ball-bearings (BBs) to track the movement of bones or surgical implants over time [10].

Two constellations of BBs are injected into the patient's pelvis prior to osteotomy: one co-located on the expected acetabular bone fragment and the other on the larger pelvis portion. The 3D locations of the BBs are reconstructed using three fluoroscopic views of the constellations. Once the acetabulum is relocated, the 3D orientation and position of the fragment is automatically calculated using a single fluoroscopic view.

Navigation systems using optical trackers impose significant storage constraints, require non-trivial administration, and are not yet commonly found across operating rooms. However, our method only requires a small BB injection device and fluoroscopy, which is already common throughout orthopaedic operating rooms for joint surgery. Therefore, we believe the fluoroscopic method according to an embodiment of the current invention is more easily deployable than other approaches relying on optical tracking technology [11-15]. Furthermore, the registration process with an optical tracker requires a certain amount of bone exposure and may become more challenging when using minimally invasive incisions [16]. Compared to existing approaches which leverage fluoroscopy [17], an embodiment of our method only requires a single fluoroscopic image per pose estimate, does not rely on any knowledge of the 3D fragment shape, and runs without user initialization in a fraction of the time.

After BB injection, this method does not require any specialized equipment or additional workflow. Moreover, the pose estimation executes quickly and automatically between fluoroscopic captures. An important clinical contribution of this method according to an embodiment of the current invention is the ability to report 3D orientation and position of the acetabular fragment, while requiring minimal modification to an existing surgical workflow. An embodiment is the first method leveraging intraoperatively constructed fiducial constellations to automatically recover point correspondences and poses of multiple objects moving non-coherently in uncalibrated single-view fluoroscopy.

Some embodiments of the invention are also applicable to tracking of artificial objects. Real-time navigational capability would be obtained by augmenting patterns of BBs or other fiducials to each object, and collecting fluoroscopic views containing both tool and pelvis BBs.

Since surgical equipment could potentially dislodge implanted fiducial objects during the acts of chiseling or drilling, intraoperative tool tracking is essential for surgeons to avoid collisions with fiducial objects (e.g., letting the surgeon “see” in 3D where they are cutting). For example, in some embodiments the pose of the osteotome and/or drill used during PAO may be estimated and reported with respect to the pelvis. This could be independent of the osteotome vendor, by taking a 3D scan of the osteotome preoperatively and then securely attaching some fiducial object to the osteotome during the surgery. Likewise, fiducials could be attached to a robotic manipulator that is moving into position for drilling/milling, using a combination of these fiducials and a 3D scan of the end effector of the robot.

Surgical implants could also be tracked in a similar manner in some embodiments. Consider the acetabular component used for total hip arthroplasty (THA), this will be analogous to the acetabular bone fragment. A collection of fiducials could be rigidly attached to the implant and a 3D scan collected. The pose of the implant with respect to the pelvis would be reported as the physician adjusts it.

Other types of fiducials beyond BBs are used in some embodiments. For example, two deformable metallic grids of wires are impressed on the bone surfaces of the fragment and ilium. Wire intersection points would be treated as point fiducials and sharp feet on the underside would facilitate insertion into bone. Each grid would be pressed against the surface of the pelvis, temporarily attached, and removed at the conclusion of the surgery. If an osteotome or K-wire were to come into contact with the grid during chiseling or drilling, the grid would most likely deform and become partially detached from the pelvis. However, removal of this larger grid should be significantly easier and lower risk, compared to the removal of a small, loose, BB.

Sets of percutaneously inserted wires with radiographic markers located at the tips of each wire also serve as temporarily affixed fiducial objects in some embodiments. Prior to osteotomy, sets of three or four wires would be percutaneously inserted into both the ilium and fragment bone regions. As each wire tip is expected to remain fixed throughout the surgery, the radiographic markers are then used as point fiducials.

### Related Work

Early navigation systems for PAO, and other pelvic osteotomies, relied on optical trackers [11, 12, 18, 19], or custom cutting guides [20,21]. These systems were limited to assistance during the osteotomies required to create the acetabular fragment, and did not provide estimates of the fragment's updated pose.

More recent systems have focused on reporting the pose of a relocated fragment [13-15, 17, 22, 23]. Fragment pose updates may be provided in real-time by directly attaching an optically tracked rigid body to the fragment as demonstrated in [13]. However, attaching a large rigid body to the acetabular region is challenging, especially when using a minimally invasive technique specialized for PAO [16]. In order to estimate fragment poses and avoid the attachment of an extra rigid body, [14] and [15] digitize specific points on the fragment with an optically tracked pointer tool after each adjustment of the fragment. This digitization adds minor overhead to the operative time in [14] and causes some ambiguity between rotation and translation in [15]. In [14], fragment pose errors ranged from 1.4-1.8° in rotation and 1.0-2.2 mm in translation.

Eliminating the need for optical tracking systems, [17] used multiple fluoroscopic views to track the acetabular fragment, ipsilateral femur, and pelvis. A multiple-component intensity-based 2D/3D registration [24] of patient anatomy was used, requiring no external objects and maintaining compatibility with any PAO approach. However, the method suffers from several limitations and constraints that interfere with a typical surgical workflow:
An approximate AP fluoroscopic view, two additional views, and manual annotation of a single anatomical landmark are required to initialize the method
Accuracy of the approach degrades as intraoperative fragment shapes differ from preoperatively planned shapes
The computation time on state-of-the-art hardware is not real-time, approximately 25 seconds.

In order to overcome these limitations, the methods according to some embodiments of the current invention leverage implanted BBs and extend RSA-related techniques to automatically track the migration of the acetabular fragment using a single view per adjustment.

Since its introduction in the 1970's, RSA has been used for a variety of applications [10], including the longitudinal analysis of orthopaedic implant migration [25], bone growth [26], and even PAO stability [27]. Recent work in the RSA community has incorporated 2D/3D registration technology to track the movement of bones and implants without relying on inserted BBs [28,29]. Similar to [17], these methods require manual input and multiple X-ray views.

The implantation of BBs for intraoperative fragment tracking during PAO was first introduced in [30] and [31]. However, this approach is not easily incorporated into a surgical workflow, since it requires: the manual identification of BB correspondences, multiple post-osteotomy views, and a calibrated CBCT C-Arm.

Several methods for automatic 3D BB reconstruction have been developed by the CBCT community [32-35]. These methods require a calibrated C-Arm, leverage more than three projections, or rely on an orbital motion constraint to help establish correspondences. For intraoperative coronary artery reconstruction, epipolar constraints have been applied to automatically prune invalid point correspondences between two [36], and three [37], fluoroscopic views. Structure-from-Motion pipelines operate in a similar fashion and use the dense correspondences found in large photographic collections to reconstruct rigid structures in 3D [38-40].

Methods using a known 3D marker constellation, two 2D X-ray views, and varying levels of manual interaction have been developed for patient positioning and motion compensation in radiation therapy [41-43].

Single plane RSA was proposed in order to avoid the less-common, bi-planar, imaging devices used in RSA [44]. However, the method requires the use of a calibration cage, does not address the establishment of 2D/3D correspondences, and was only evaluated for single object pose recovery.

Automatic pose and correspondence estimation between a single rigid collection of BBs and one fluoroscopic view was described in [45] and [46]. The poses of custom tailored fiducial objects using lines and ellipses may also be computed automatically in a single view [47, 48]. These poses are generally restricted to tracking the relative motion of the C-Arm, since the relationship between the patient's anatomy and the intraoperatively inserted fiducial is typically unknown.

The method of [45] was incorporated into fluoroscopic systems for estimating the poses of multiple BB constellations required for knee kinematics [49, 50]. However, the mechanism used to identify constellation membership and establish 2D/3D correspondences, was not described.

The pipeline according to an embodiment of the current invention is able to accurately, quickly, and automatically provide pose estimates of a relocated bone fragment during PAO. No reliance on external tracking devices is required. Furthermore, the pose estimation method does not require: a calibrated C-Arm, multiple-views, a specific constellation pattern, accurate knowledge of the fragment shape, or any manual establishment of correspondence.

### EXAMPLES

The following describes some specific examples according to some embodiments of the current invention. The general concepts of this invention are not limited to these particular examples.

### Materials and Methods

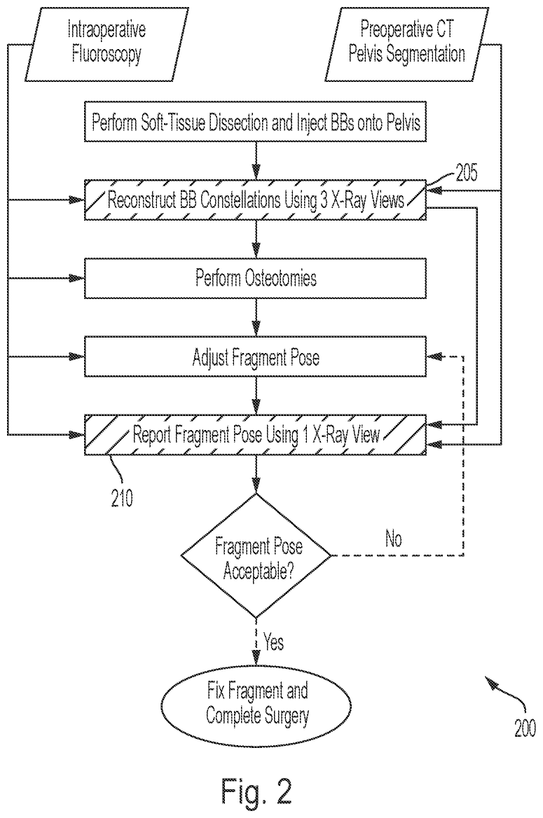

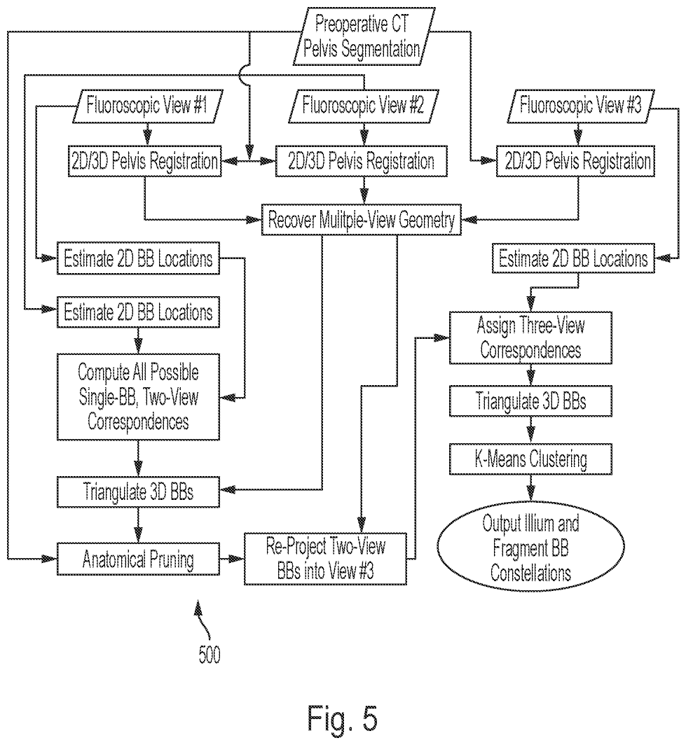

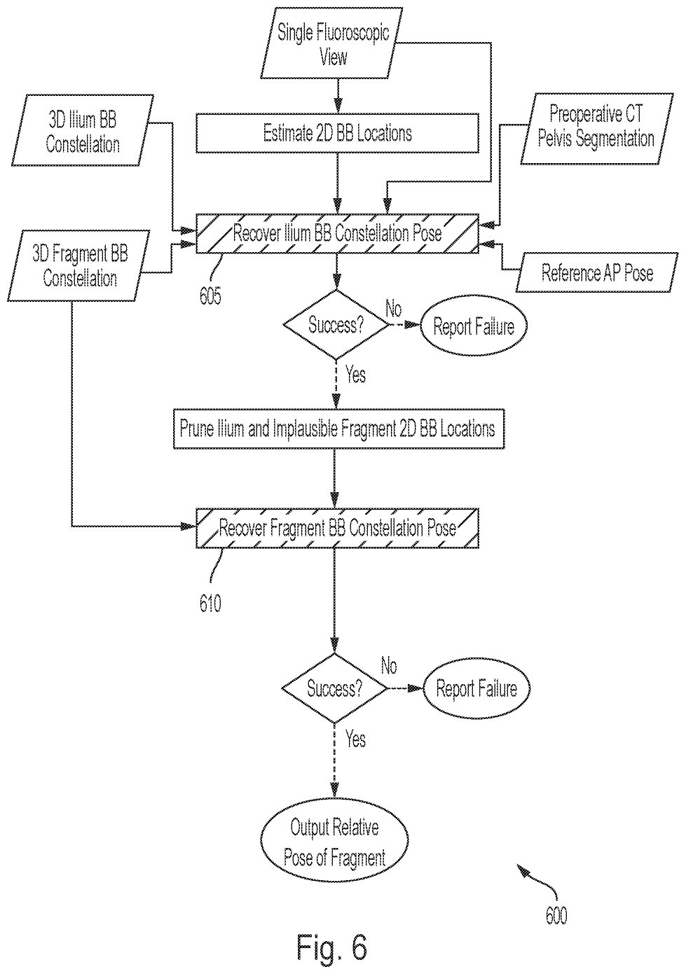

An embodiment of the current invention requires some preoperative processing and two distinct phases during the surgery. CT scanning, segmentation of the anatomy, and anatomical landmark digitization make up the preoperative processing. The first intraoperative phase is performed only once and includes BB injection and reconstruction. Pose estimation of the acetabular fragment from a single fluoroscopic view represents the second intraoperative addition. In order to achieve the desired amount of femoral head coverage, it is typical for a surgeon to iterate between collecting fluoroscopy and adjusting the fragment. Therefore, our processing combines intelligent pruning and GPU acceleration to avoid any significant delay to the workflow. FIG. 2 shows the workflow 200 of the method at a high level. The key contributions of this work are the BB reconstruction 205 and single-view pose estimation components 210, which are highlighted in gray. Full details of the preoperative processing, intraoperative BB reconstruction, and intraoperative fragment pose estimation are now provided, with reference to FIG. 2. Full workflows for the reconstruction and pose estimation components are shown in FIGS. 5 and 6, respectively.

### Preoperative Processing

Preoperative processing can proceed identically to that in [17], which we briefly describe here. A lower torso CT scan is obtained and resampled to have 1 mm isotropic voxel spacing. An automated method [51] is used for an initial segmentation of the pelvis and femurs; any inconsistencies around the femoral head and acetabulum are cleaned up manually. Anatomical landmarks are manually annotated to define the anterior pelvic plane (APP) coordinate system [52], and for later use as initialization of pre-osteotomy pelvis registrations. The origin of the APP is set at the center of the ipsilateral femoral head, and the mapping from APP coordinates to the CT volume coordinates is denoted by T^{V}_{APP}. Six additional landmarks are manually annotated in order to create a planned fragment shape, which is only used to visualize the intra-operative movement of the fragment.

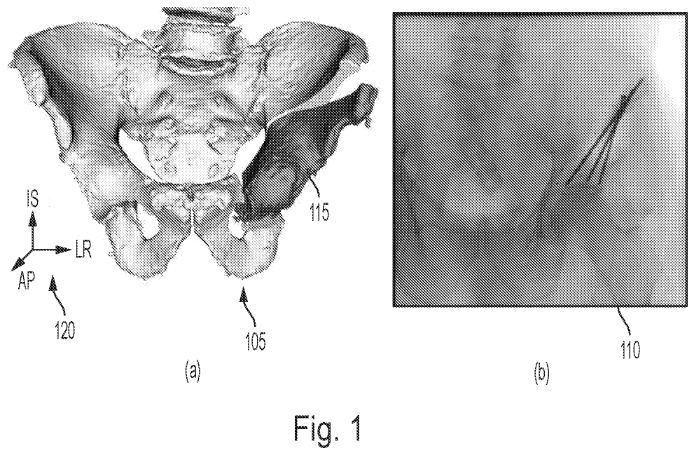

Examples of the APP axes orientation and a planned fragment shape are shown in FIG. 1. The fragment pose 105 shown in (a) was estimated using the view 110 shown in (b). A precise model of the acetabular fragment 115 is not required by the method according to this embodiment; the 3D bone surfaces in (a) were constructed using a preoperative plan of the osteotomies. The anatomical axes 120 of the anterior pelvic plane are also shown in (a); left/right (LR) as X-axis, inferior/superior (IS) as Y-axis, and anterior/posterior (AP) as Z-axis.

A precise model of the acetabular fragment is not required by the method according to this embodiment; the 3D bone surfaces in (a) were constructed using a preoperative plan of the osteotomies. The anatomical axes of the anterior pelvic plane are also shown in (a); left/right (LR) as X-axis, inferior/superior (IS) as Y-axis, and anterior/posterior (AP) as Z-axis.

### Intraoperative BB Reconstruction

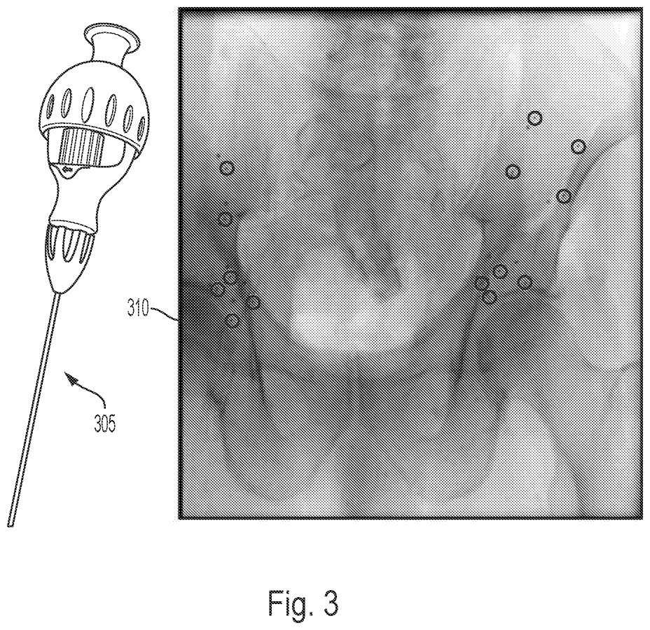

FIG. 3 illustrates, on the left, a Halifax bead injection device 305 used in some embodiments in four of the cadaver surgeries. The device is used to implant two, four-BB constellations onto the ipsilateral side of the patient's pelvis, with one constellation lying on the area expected to lie on the acetabular fragment and the other on the larger pelvis fragment. The BBs are injected after performing soft-tissue dissection, but prior to osteotomy. On the right, a pre-osteotomy fluoroscopic image 310 is shown with automatic detections of injected beads highlighted by yellow circles; every injected BB was detected. The larger BBs were used to help establish the ground truth pose of the fragment and as such, are not used and not detected during intraoperative pose estimation. Photograph of injector from: https://halifaxbiomedical.com.

Three distinct fluoroscopic views are collected while the patient anatomy remains stationary. For each view, the radial symmetry algorithm [53] is used to automatically locate each BB in 2D. The 3D locations of each BB are constructed by recovering the relative pose information of each view, establishing inter-view BB correspondences, and performing triangulation [54].

Using the strategy laid out in [17], relative poses between the three views are recovered by performing 2D/3D rigid registrations of the patient's preoperative pelvis to each view. Since some of our pre-osteotomy views have excessive pelvic tilt and violate the approximate AP view assumption, we select more than the single landmark described in [17] for initialization of the pipeline. Appendix A describes the parameters used for the intensity-based registrations.

BB correspondences are automatically established using a combination of anatomical information and the multiple-view geometry between the three C-Arm poses. Two of the views are selected to create a candidate set of two-view, single-BB, correspondences and triangulated 3D points. Although we have made no assumptions about the geometry of these views, one of the views was always an approximate AP orientation with a variable amount of pelvic tilt.

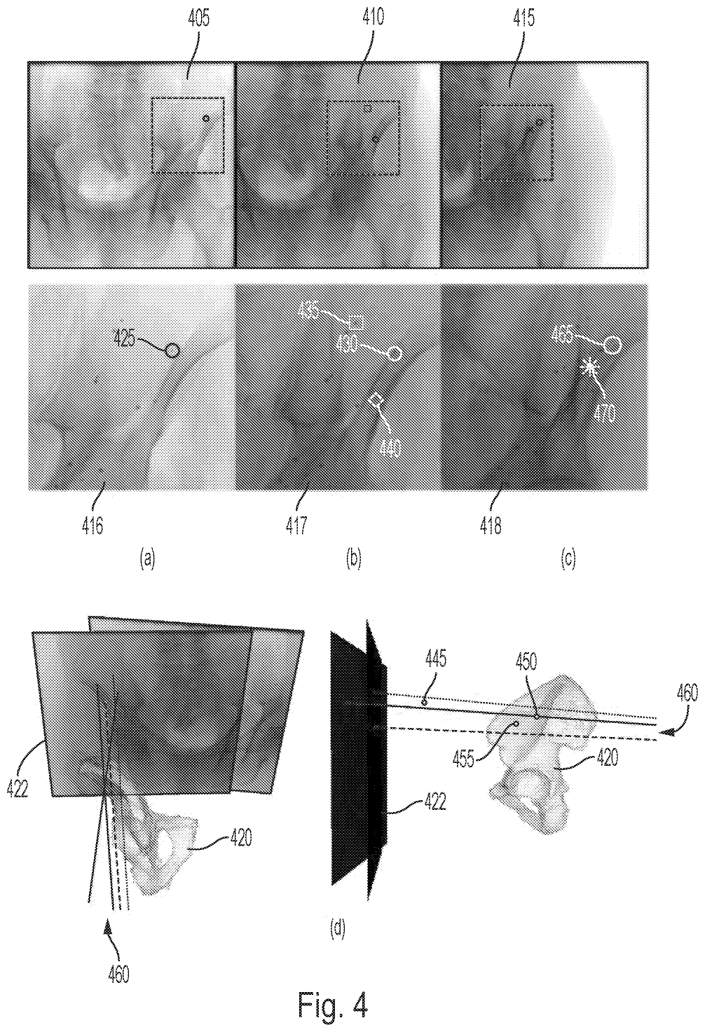

The candidate correspondences are created by first considering all possible combinations of single-BB correspondences between the two views and pruning candidates that result in a triangulated point located more than 10 mm away from the pelvis surface. The red sphere shown in FIG. 4 (discussed in more detail below) is an example of a correspondence pruned in this way. Candidate three-view correspondences are constructed by pairing each of the remaining two-view correspondences with every 2D BB detection in the third view. For each candidate three-view correspondence, the two-view 3D triangulation is re-projected into the third view and the distance to the hypothesized 2D match is recorded. Intuitively, re-projection distances for valid correspondences should be smaller than distances from invalid matches, as shown with the green and yellow re-projections in FIG. 4. Correct three-view correspondences are established by greedily selecting the candidate correspondences with minimum re-projection distances in the third view. The final 3D reconstructions are triangulated using the correct three-view correspondences. In this way, the third view is used to enforce consistency and refine the 3D triangulation. A visual example is shown in FIG. 4 and a more formal description is in Appendix B.

After pruning reconstructed BBs on the contralateral side, the process is completed by classifying the remaining BBs as fragment/non-fragment using a K-Means clustering of the BB positions (K=2).

The workflow of the entire reconstruction process is shown in FIG. 5, which is described in more detail below. Annotation speed and computation time is not a critical factor at this point in the procedure, since the BB constellations are not required until the fragment has been relocated; osteotomies may be performed immediately after the three fluoroscopic views are obtained.

### Intraoperative Pose Estimation

After osteotomies have been performed and the acetabular fragment has been relocated, a single fluoroscopic image may be used to recover the fragment's pose with respect to the APP, Δ_{APP}. Once the poses of the ilium and fragment BB constellations with respect to the C-Arm, T_{C}^{IL} and T_{C}^{FR}, are recovered, Δ_{APP} is computed as in (1).

Δ_{APP}=T_{V}^{APP}T_{C}^{FR}T_{IL}^{C}T_{APP}^{V} (1)

FIG. 4 illustrates a visual example of the pre-osteotomy reconstruction process for a single BB. Three fluoroscopic views 405, 410, 415 used for reconstruction are shown in (a), (b), and (c). The initial two-view triangulations are derived from (a) and (b), while (c) is used for re-projections of initial triangulations. Regions pertinent to this example are indicated by yellow boxes 416-418 and are magnified in the bottom row. 3D renderings 420 of the patient's ipsilateral hemi-pelvis and the relative location of the C-Arm detector 422 for the first two views are shown in (d). The green circle 425 in (a) indicates the location of a detected BB, whose 3D location is to be reconstructed. In (b), the green circle 430 shows the detected BB location with true correspondence to BB in (a); the red square 435 and yellow diamond 440 show detected locations with incorrect correspondence. In (d), the three colored spheres are initial triangulations of the BB from (a) when matched with the BBs of varying colors in (b). The red sphere 445 is not located on the pelvis and its candidate correspondence is pruned. However, the green 450 and yellow spheres 455 are located on the pelvis and must be checked using (c). Lines 460 between the X-ray source and BB locations on the detector are colored consistently with (a), (b), and (c); note the intersection between the green lines. The green circle 465 in (c) indicates the detected location of the BB in true correspondence with the green circles 425 and 430 in (a) and (b). The green “X” within the circle 465 is the re-projection of the green sphere 450 from (d) and the yellow asterisk 470 is the re-projection of the yellow sphere 455. Since the green sphere 450 was triangulated using a correct correspondence, its re-projected distance to the BB detection in (c) is very small compared to the re-projected distance of the yellow sphere 455, which was triangulated using an incorrect correspondence.

Since the BB constellations are constructed in the original pelvis volume coordinate frame, the composition of T_{C}^{FR}T_{IL}^{C} is valid and maps points on the preoperative fragment region to their adjusted locations. Using Δ_{APP}, the current pose of the fragment may be visualized (FIG. 1) and pose parameters or biomechanical (e.g. LCE) angles may also be displayed. FIG. 6 depicts the entire, end-to-end, pose estimation workflow.

As was the case for each fluoroscopic view used for pre-osteotomy BB reconstruction, the radial symmetry algorithm is used to detect each BB in the 2D fluoroscopic image automatically. Since the 3D/2D BB correspondences are not yet established, it is not feasible to directly apply classic PnP approaches [54] for calculation of T_{C}^{IL} or T_{C}^{FR}. Since manual identification is tedious and error-prone, an automatic method to establish correspondences is the appropriate intraoperative strategy. One possible, although naïve, approach would be to enumerate over all possible correspondences and their poses. Digitally reconstructed radiographs (DRRs) and similarities with the fluoroscopic image would be computed for each candidate pose, with the actual pose implied by the best similarity score. There are 1,680 possible correspondences when all 8 BBs are detected in the view. Since screws and K-wires are used to fix the fragment, false BB detections are common and may result in 12 BB detections, yielding 11,880 possible correspondences. For bilateral cases, with BBs, screws, or K-wires on the contralateral side, 24 BB detections could be possible, yielding 255,024 possible correspondences. Examples of 2D BB detections in fluoroscopy images are shown in Appendix D, with the number of BB detections per image varying between 7 and 21. The sheer number of possible poses precludes the brute-force strategy from working in an intraoperatively compatible timeframe. However, we shall describe a procedure for pruning the number of candidate poses by several orders of magnitude, enabling the required similarity scores to be intraoperatively computed through GPU acceleration.

FIG. 5 illustrates a workflow overview 500 of the intraoperative BB reconstruction process. Three separate 2D/3D pelvis registrations of each fluoroscopic view are performed to recover the relative poses of the C-Arm. Triangulations from all possible single-BB correspondences in the first two views are computed and pruned using the 3D pelvis segmentation. Any remaining, invalid, correspondences are eliminated by re-projecting into the third view and checking for consistency with 2D BB detections. Using the correct three-view correspondences, the BBs are re-triangulated, and K-Means is used to label each BB as belonging to the ilium or fragment constellation.

For a given 4-BB constellation, the general pruning strategy enumerates over each 3-BB sub-constellation. Furthermore, the full set of possible 3-BB 3D/2D correspondences for each sub-constellation is examined. Potential solutions to the P3P problem [55] are considered for each set of correspondences. Since we are concerned with pose estimation using fluoroscopic imagery, our approach to the P3P problem assumes that the BB constellation lies between the X-ray source and detector. This assumption enables solutions to be ignored which: are impossible given the rigid structure of the constellation, or which place the BB constellation too close to the X-ray source. Many poses that would be produced from incorrect 3-BB correspondences are discarded in this way. In addition to the point sets and hypothesized correspondences, a set of source-to-detector ratios is also required as input to the P3P solver. The source-to-detector ratios are used to back-project one of the 2D BB detections to possible 3D locations, simplifying the pruning problem. Full details of this approach are described in Appendix C. Solutions reported by the P3P solver are further pruned according to anatomical constraints. The candidate source-to-detector ratios and anatomical constraints differ for the ilium and fragment BB constellations.

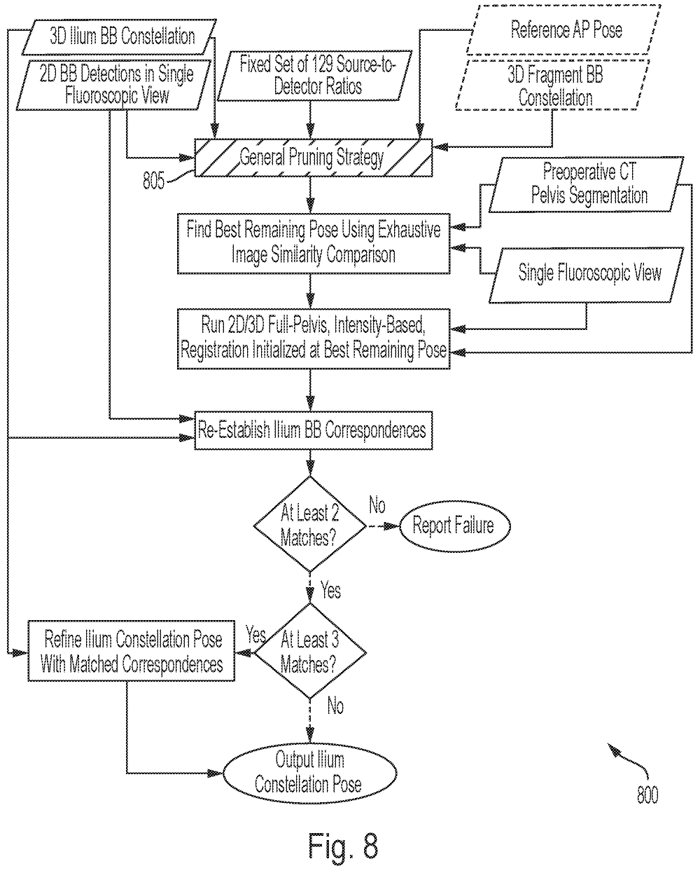

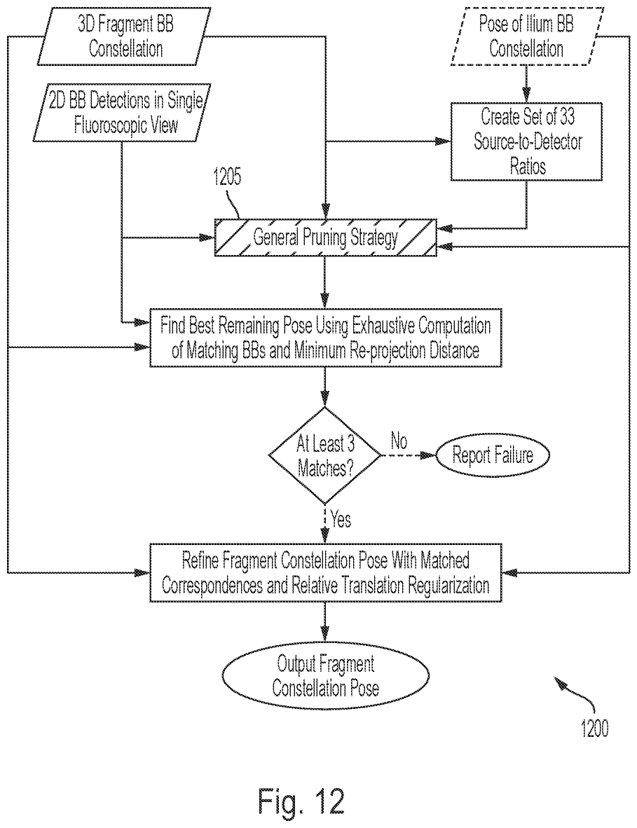

FIG. 6 illustrates a complete workflow 600 used in some embodiments for single-view relative pose estimation of the acetabular fragment. Gray-shaded boxes correspond to the ilium 605 and fragment BB constellation 610 pose estimate workflows described in FIGS. 8 and 12, respectively. The relative pose of the bone fragment is calculated using the BB constellation poses.

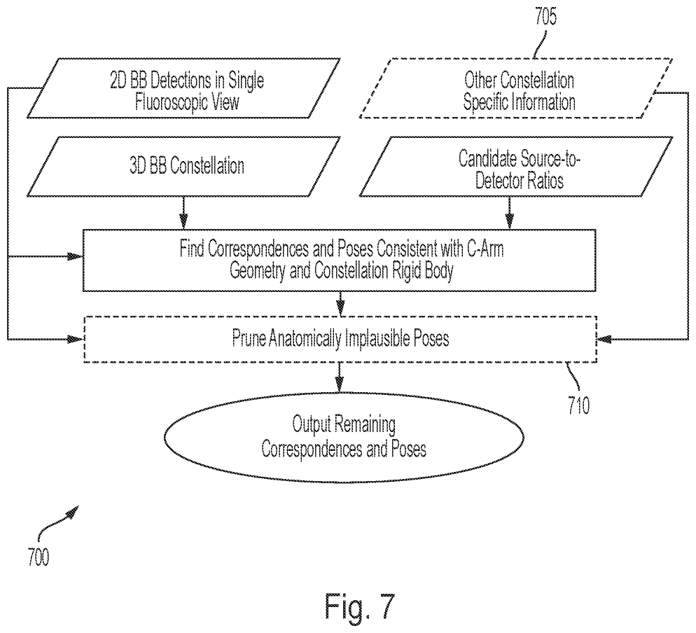

FIG. 7 illustrates the data flow 700 of the general pruning strategy used during BB constellation pose estimations in some embodiments. Dashed boxes 705 and 710 indicate input data and processing that will be specific for either ilium or fragment processing.

The pose of the ilium BB constellation is recovered first and is then used to assist with establishing the pose of the fragment BB constellation. FIG. 8 shows the high level data flow 800 for pose estimation of the ilium constellation. The workflow of the general pruning strategy 805 (corresponding to FIG. 7) is re-used here and highlighted in gray, with inputs specific to ilium pruning emphasized by dashed borders. Since the general pruning strategy returns multiple possible poses and BB correspondences, image intensities are used to select the best candidate pose. The pose is further refined by a 2D/3D intensity-based registration of the pre-osteotomy pelvis, with success criteria automatically verified by the number of ilium BBs matched through re-projection.

A set of 129 uniformly spaced source-to-detector ratios is used for each ilium P3P invocation: {0.6+0.003125k|k=0, 1, . . . , 128}. Using the APP coordinate frame, a reference AP orientation of the pre-osteotomy pelvis, with respect to the C-Arm, is constructed and used for pruning anatomically implausible ilium poses. The AP orientation has the following properties: the patient is supine with the X-ray detector placed anteriorly, the AP axis is parallel to the C-Arm depth axis, the IS axis is parallel to the 2D image row axis with the top of the image more superior than the bottom, and the LR axis is parallel to the 2D image column axis. Each candidate P3P pose is examined to obtain the difference in orientation from the reference AP pose and an Euler decomposition is used to obtain rotation angles about each anatomical axis. Poses are pruned when the magnitude of any Euler angle is greater than 60°. Using such a large range of allowable angles permits all reasonable C-Arm orientations while eliminating highly unlikely poses, such as those that place the detector beneath, or nearly orthogonal with, the surface of the operating table.

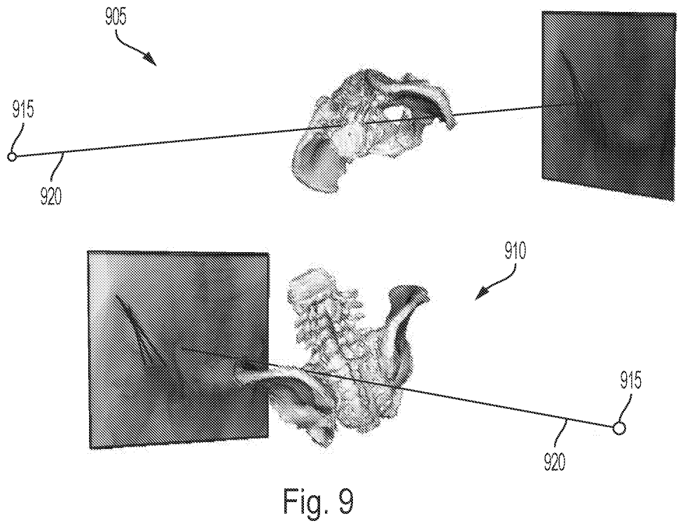

An example of a pose pruned for excessive difference from the reference AP orientation (137° about the AP axis, in this case) is shown in the top row 905 of FIG. 9. Using each remaining candidate ilium pose, the original fragment BB constellation is projected into the view; e.g. where the fragment BBs would be located in 2D had the fragment not been moved. Since the majority of fragment movement consists of rotation, the re-projected fragment BBs should lie nearby to 2D BB detections. Poses are pruned when less than 3 of the fragment BBs are projected inside the bounds of the 2D image. For each projected fragment BB, the distance to the nearest 2D detection is calculated, and the three BBs with the smallest nearest distances are recorded. When the mean distance associated with these three BBs is greater than 200 pixels the candidate ilium pose is pruned. For example, the bottom row 910 of FIG. 9 corresponds to a pose pruned due to a large mean fragment BB constellation re-projection distance (297 pixels). For each example, the candidate correspondences were able to satisfy the constraints of the P3P solver. However, the implausibility of each pose reveals the incorrectness of the correspondences. The green sphere 915 indicates the X-ray source with a green line 920 connecting to the principal point on the X-ray detector.

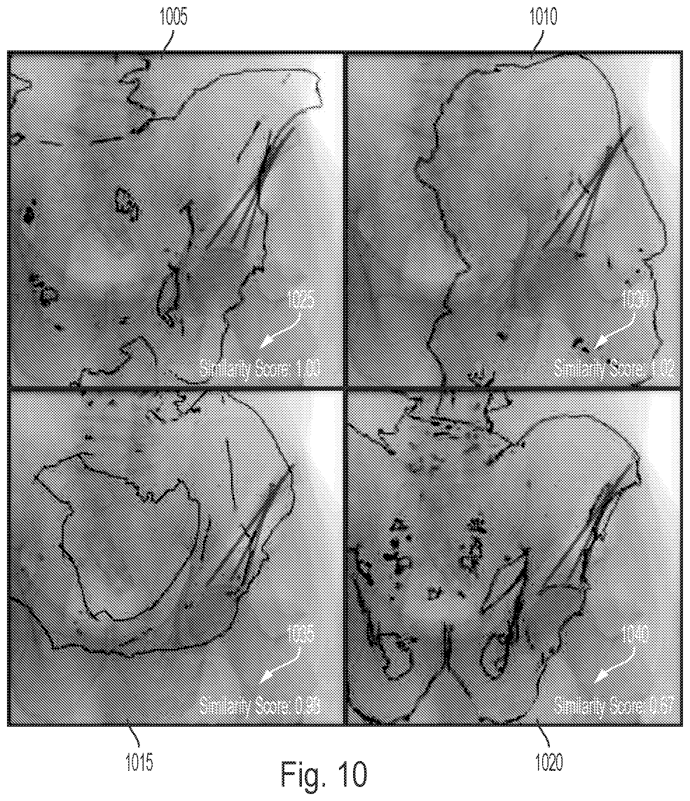

Once the general pruning strategy has been completed for the ilium BB constellation, the naïve brute-force, intensity-based, approach is used to select the best ilium pose from the remaining candidates. FIG. 10 shows several examples 1005-1020 of image similarity calculated from 4 poses derived from different correspondences. Green edges, derived from a specific pelvis pose, are overlaid over the intraoperative fluoroscopic image. Agreement between the overlaid edges and base image indicates agreement between the hypothesized pose and true pose. Image similarity scores 1025-1040 are listed in the bottom right of each overlay. The scores are computed from DRRs, computed at each candidate pose, and the intraoperative fluoroscopic image. Lower scores indicate better similarity, with the bottom right example 1040 representing the most likely pose of the four. This pose is used as initialization for an intensity-based 2D/3D registration of the pre-osteotomy pelvis to the fluoroscopic image. Details of the intensity-based registration parameters are listed in Appendix A.

Using the pose estimate computed during the intensity-based registration, final ilium BB correspondences are established by re-projecting the 3D ilium BBs into 2D. Correspondences are greedily assigned based on the minimum 2D distances between projected BB locations and detected 2D BB locations. However, no correspondence is established for projected BBs with minimum distances greater than 10.5 pixels. When less than two correspondences are established, we consider the algorithm to have failed in establishing the ilium pose and no further processing is performed. The ilium pose is set to the intensity-based registration pose when exactly two correspondences are established. When three or four correspondences are established, the ilium pose is refined by optimizing over the corresponding ilium BB re-projection distances starting from the intensity-based pose as the initial guess.

The set of 2D BB detections is pruned down to exclude: BBs already matched to the ilium, and any BBs that are distant from the expected location of the fragment. A BB is considered distant if the closest, re-projected, fragment BB is greater than 200 pixels away. This is a variation of the process previously used for pruning ilium poses by re-projection of 3D fragment BBs.

The fragment pose recovery is started by conducting the general pruning strategy over candidate fragment BB correspondences and poses. Since approximate depth of the BBs is known from the ilium pose recovery process, only 33 source-to-detector ratios are passed to the P3P solver. A reference source-to-detector ratio, \hat{r}, is is computed by mapping the centroid of the fragment 3D BB constellation into the C-Arm coordinate frame using the ilium pose. The source-to-detector ratios are then uniformly sampled about this reference: {\hat{r}±0.003125k|k=0, 1, . . . , 16}. Using each solution produced by the P3P solver, the relative pose of the fragment is computed using (1). Any relative pose with rotation magnitude greater than 60° or translation magnitude greater than 30 mm is pruned.

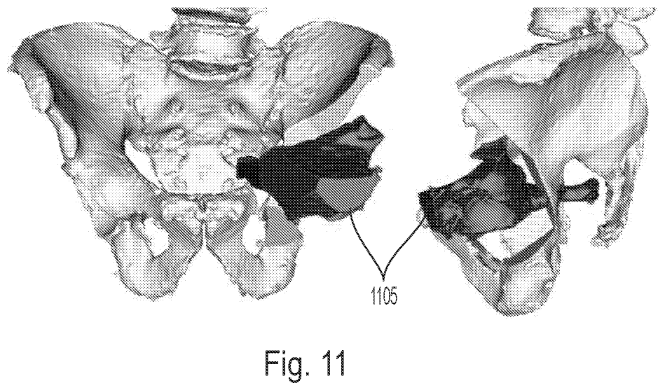

FIG. 11 illustrates an example of an implausible fragment pose 1105 which was pruned due to a large rotation of 142°. As with the examples in FIG. 9, the candidate correspondences, despite their incorrectness, were able to satisfy the P3P solver constraints.

Due the difficult nature of the chiseling process, the true shape of the acetabular fragment usually differs from the preoperatively planned shape. For this reason, image similarities are not used to select the best candidate returned from the general pruning process. Instead, the best candidate is selected by choosing the pose yielding the largest number of matching BBs and the smallest mean re-projection distance. The match criterion used for ilium matches is reused here. When less than 3 BBs are matched, the method reports failure. However, the fragment pose is refined by an optimization over re-projection distances if at least 3 BBs are matched. The optimization is regularized by the translation magnitude of the fragment pose relative to the APP. This regularization is reasonable, since the fragment movement is believed to consist primarily of rotation and the approximate depth is known from the ilium pose.

FIG. 12 illustrates a workflow 1200 of the fragment BB constellation pose estimation process. Gray shading 1205 corresponds to the invocation of the general pruning strategy (FIG. 7), with the ilium pose used to prune implausible relative fragment poses. The best pose returned by the general strategy is selected by maximizing the number of matched re-projected fragment BBs with smallest mean in-plane, re-projection, distance. The final pose is only reported when at least three fragment BBs are matched.

It is important to note that the approach described here only requires correspondences to be established for three ilium BBs and three fragment BBs. Therefore, the proposed method provides some robustness to occlusion, since it is unlikely that more than one BB from a single constellation will be occluded for any given view. Likewise, it is still feasible to obtain fragment pose estimates when a single BB (per constellation) becomes dislodged from the bone.

### Cadaver Experiments

Surgeries performed on three, non-dysplastic, cadaveric specimens were used to evaluate the proposed method. Specimens 1, 2, and 3 were aged 89, 87, 94 and were male, female, and male, respectively. Preoperative processing and planning was performed bilaterally for each specimen and six PAOs were performed by a PAO surgeon. The Halifax injector, using BBs of 1 mm diameter, was only used during surgeries for specimens 2 and 3. For specimen 1, bone burs were created on the surface of the pelvis, and 1.5 mm diameter BBs were affixed with cyanoacrylate. The larger BBs were also inserted into specimens 2 and 3, but were only used for ground truth calculations. A comprehensive discussion of BB insertion and the fragment pose ground truth protocol is found in [17]. Ground truth poses for specimen 1 were calculated using the 2D/3D known BB constellation approach, whereas specimens 2 and 3 use the 3D/3D method.

Three fluoroscopic views were used to reconstruct the pre-osteotomy 3D BB constellations for each surgery. Pose estimation of the relocated fragment was conducted on 3 separate fluoroscopic images, each with different viewing geometries. All fluoroscopy was obtained using a Siemens CIOS Fusion C-Arm with 30 inch flat panel detector.

### Results

For pre-osteotomy BB reconstruction, there were no missed detections in the 2D images and a single false detection in one image. Table 1 summarizes the reconstruction errors. The mean reconstruction error of the larger BBs implanted into specimen 1 was 2.6 mm. For specimens 2 and 3, the mean reconstruction error of the smaller, injected, BBs was 1.4 mm. The mean computation time for the entire reconstruction pipeline was 8.3±0.4 seconds. When excluding the 2D/3D full pelvis registration time required for relative pose recovery of the C-Arm, the correspondence establishment and reconstruction took 0.7±0.1 seconds. Timing measurements were conducted using a single NVIDIA P100 (PCIe) GPU and seven cores of an Intel Xeon E5-2680 v4 CPU.

TABLE 1
A summary of BB reconstruction errors for each surgery, specified
by the cadaver specimen number and operative side. The means
and standard deviations of reconstruction errors are given for
the separate ilium and fragment BB constellations and also the
entire set of BBs. For each surgery, four BBs were reconstructed
for each of the ilium and fragment constellations.

| | Reconstruction Errors (mm) | | |
|---|---|---|---|
| Surgery | Illium BBs | Fragment BBs | All BBs |
| 1 Left | 2.5 ± 0.4 | 3.2 ± 0.2 | 2.9 ± 0.5 |
| 1 Right | 2.1 ± 0.2 | 2.7 ± 0.5 | 2.4 ± 0.5 |
| 2 Left | 1.6 ± 0.3 | 1.4 ± 0.2 | 1.5 ± 0.3 |
| 2 Right | 1.3 ± 0.5 | 1.1 ± 0.1 | 1.2 ± 0.3 |
| 3 Left | 1.3 ± 0.3 | 1.1 ± 0.4 | 1.2 ± 0.3 |
| 3 Right | 1.4 ± 0.2 | 1.6 ± 0.2 | 1.5 ± 0.2 |

A summary of the maximum number of ilium and fragment poses considered, and the actual poses considered due to pruning, is shown in Table 2. Due to the range of source-to-detector distances searched over, the maximum number of poses considered is greater than the maximum number of possible correspondences. On average, 99.6% of the maximum number of ilium poses are pruned by the P3P solver step. Using anatomical constraints, the remaining poses are pruned an average of 95.9%. The maximum number of poses are pruned by an average of 97.3% for the fragment case, with anatomical constraints pruning 81.2% of the remaining poses on average.

Pose estimation was successfully performed on 18 total views (3 per surgery). Errors in rotation were below 3° for 12 of the 18 cases, with a mean of 2.4°. When the rotation errors were decomposed about anatomical axes, only rotation about the IS axis had errors greater than 3°. In terms of both mean and standard deviation, rotation measurements about the AP axis were the most accurate, followed by LR, and then IS. The maximum 3D LCE angle error was 1.8° and the mean was 1.0°. The mean translation error was 2.1 mm, and was less than 3 mm for 15 of the 18 estimates. Mean translation errors about the anatomical axes were all within 0.2 mm of each other, and the maximum difference between standard deviations was 0.3 mm. The entire listing of errors for each pose estimate is shown in Table 3.

Table 4 includes a full summary of the number of BB detections and matches in each image. All four ilium BBs were matched in 6 of the 18 cases and all four fragment BBs were matched in 16 of the 18 cases. The mean rotation, translation, and LCE angle errors for estimates with 4 ilium BBs matched were 1.7°, 2.1 mm, and 1.0°, respectively. With less than 4 ilium BBs matched, the mean errors were 2.8°, 2.0 mm, and 1.1°, respectively. With 4 fragment BBs matched, the mean rotation, translation, and LCE angle errors were 2.3°, 2.1 mm, and 1.0°, respectively. The mean errors were 3.1°, 1.5 mm, and 1.6°, when less than 4 fragment BBs matched.

TABLE 2
A summary of the number of pose and correspondence combinations for the ilium and fragment
BB constellations during the process of single-view fragment pose estimation for the
three different views of each cadaver surgery. The maximum number of possible combinations
are listed, along with the number after each pruning step. Each of the pose candidates
after anatomical pruning for the ilium is used for initialization of the full-pelvis
intensity-based 2D/3D registration. The maximum number of fragment poses and correspondences
is lower than that of the ilium, since the ilium correspondences are established first
and implausible fragment BB detections are pruned.

| | | # Ilium Pose/Correspondence Candidates | | | # Fragment Pose/Correspondence Candidates | | |
|---|---|---|---|---|---|---|---|
| | | Before | After P3P | After Anat. | Before | After P3P | After Anat. |
| Surgery | Proj. | Pruning | Pruning | Pruning | Pruning | Pruning | Pruning |
| 1 Left | 1 | 885,456 | 2,567 | 155 | 7,920 | 139 | 34 |
| 1 Left | 2 | 681,120 | 2,381 | 80 | 7,920 | 157 | 40 |
| 1 Left | 3 | 4,117,680 | 6,230 | 168 | 15,840 | 198 | 22 |
| 1 Right | 1 | 510,840 | 843 | 24 | 15,840 | 178 | 33 |
| 1 Right | 2 | 260,064 | 645 | 17 | 7,920 | 97 | 18 |
| 1 Right | 3 | 371,520 | 472 | 14 | 7,920 | 153 | 52 |
| 2 Left | 1 | 108,360 | 450 | 12 | 3,168 | 68 | 9 |
| 2 Left | 2 | 510,840 | 590 | 10 | 3,168 | 89 | 6 |
| 2 Left | 3 | 1,126,944 | 1,204 | 10 | 3,168 | 89 | 10 |
| 2 Right | 1 | 108,360 | 1,097 | 70 | 3,168 | 156 | 30 |
| 2 Right | 2 | 371,520 | 858 | 23 | 3,168 | 142 | 17 |
| 2 Right | 3 | 885,456 | 1,269 | 32 | 3,168 | 106 | 22 |
| 3 Left | 1 | 371,520 | 783 | 35 | 3,168 | 122 | 12 |
| 3 Left | 2 | 1,408,680 | 1,359 | 48 | 792 | 12 | 4 |
| 3 Left | 3 | 173,376 | 958 | 50 | 3,168 | 100 | 12 |
| 3 Right | 1 | 173,376 | 1,639 | 129 | 3,168 | 106 | 22 |
| 3 Right | 2 | 108,360 | 1,139 | 85 | 3,168 | 108 | 17 |
| 3 Right | 3 | 260,064 | 698 | 58 | 792 | 26 | 5 |

In the third view for the left side of specimen 1, one ilium BB was outside the image bounds and not detected. On the left side of specimen 2, one of the ilium BBs was occluded by K-wire in each view and therefore not detected. Analysis of the postoperative CT revealed that this BB was actually dislodged by either: performance of the ilium osteotomy or insertion of the K-wire. The missed ilium detections in views 1 and 2 on the right side of specimen 2, were occluded by screws. Occlusion by K-wire also caused the missed ilium detection in view 2 on the right side of specimen 3. However, according to the postoperative CT this ilium BB was also displaced from the bone. The missed fragment BB detections were caused by K-wire occlusion.

The mean computation time for the single-view pose estimation was 0.7±0.2 seconds, and was measured using the same hardware used to record reconstruction times.

Thumbnails of each fluoroscopy image used for fragment pose estimation during the cadaver experiments are found in Appendix D.

TABLE 3
A summary of the single-view fragment pose and lateral center edge (LCE) angle errors. Errors are reported
for the three fluoroscopic views taken during each surgery, identified with a cadaver specimen number and
operative side, along with the means and standard deviations over all surgeries. In addition to the rotation
and translation pose error magnitudes, a full decomposition of pose errors about anatomical axes is listed.

| | | Rotation Errors (°) | | | | Translation Errors (mm) | | | | |
|---|---|---|---|---|---|---|---|---|---|---|
| Surgery | Proj. | Total | LR | IS | AP | Total | LR | IS | AP | LCE (°) |
| 1 Left | 1 | 1.7 | 0.3 | 1.5 | 0.7 | 3.1 | 2.9 | 0.7 | 1.0 | 1.0 |
| 1 Left | 2 | 2.5 | 2.2 | 0.5 | 1.1 | 1.9 | 1.3 | 0.1 | 1.3 | 0.8 |
| 1 Left | 3 | 1.6 | 0.5 | 1.3 | 0.7 | 3.2 | 3.1 | 0.9 | 0.5 | 0.9 |
| 1 Right | 1 | 1.9 | 1.9 | 0.3 | 0.1 | 2.0 | 1.6 | 1.0 | 0.5 | 0.1 |
| 1 Right | 2 | 3.3 | 0.7 | 2.8 | 1.5 | 2.0 | 0.8 | 1.2 | 1.3 | 1.4 |
| 1 Right | 3 | 2.8 | 2.6 | 0.1 | 1.1 | 2.4 | 1.5 | 1.8 | 0.1 | 0.9 |
| 2 Left | 1 | 3.7 | 1.4 | 3.3 | 0.7 | 2.3 | 1.8 | 1.4 | 0.4 | 1.3 |
| 2 Left | 2 | 3.3 | 1.1 | 3.1 | <0.1 | 1.6 | 0.7 | 1.0 | 1.1 | 0.6 |
| 2 Left | 3 | 2.2 | 0.6 | 2.1 | 0.2 | 1.7 | 0.3 | 1.6 | 0.3 | 0.8 |
| 2 Right | 1 | 2.4 | 0.9 | 2.2 | 0.4 | 1.1 | 0.1 | 0.2 | 1.1 | 1.8 |
| 2 Right | 2 | 1.7 | 0.3 | 1.6 | 0.4 | 2.1 | 0.9 | 0.3 | 1.9 | 1.1 |
| 2 Right | 3 | 3.2 | 0.5 | 3.1 | 0.2 | 1.9 | 0.6 | 0.4 | 1.7 | 1.7 |
| 3 Left | 1 | 0.7 | 0.4 | 0.1 | 0.5 | 3.0 | 2.0 | 1.9 | 1.3 | 0.4 |
| 3 Left | 2 | 1.3 | 0.6 | 0.9 | 0.6 | 1.3 | 1.1 | 0.7 | 0.1 | 1.8 |
| 3 Left | 3 | 1.2 | 1.1 | <0.1 | 0.5 | 2.2 | 0.6 | 1.7 | 1.3 | 1.8 |
| 3 Right | 1 | 2.4 | 2.3 | 0.1 | 0.2 | 1.6 | 0.6 | 0.9 | 1.2 | 0.6 |
| 3 Right | 2 | 3.0 | 2.7 | 1.3 | 0.3 | 2.3 | 0.7 | 0.7 | 2.1 | 0.3 |
| 3 Right | 3 | 5.0 | 2.7 | 3.6 | 2.0 | 1.7 | 0.8 | 0.9 | 1.2 | 1.4 |
| All | — | 2.4 ± 1.0 | 1.3 ± 0.9 | 1.6 ± 1.2 | 0.6 ± 0.5 | 2.1 ± 0.6 | 1.2 ± 0.8 | 1.0 ± 0.5 | 1.0 ± 0.6 | 1.0 ± 0.5 |

### Discussion

Although one third of fragment pose estimates had rotation errors larger than 3°, LCE angle errors were well below the 3° success criteria identified in [17]. This indicates that the proposed method is able to quantify the amount of femoral head coverage, resulting from an intraoperatively relocated acetabulum, within clinically acceptable error thresholds.

Given the automatic nature of the method and the relatively quick runtime, it should be feasible for clinicians to smoothly move between making pose adjustments to the fragment, taking fluoroscopic shots, and receiving feedback regarding the current pose estimate.

The mean rotation error when less than the full number of BBs were matched in either constellation, was 1.1° greater than the mean rotation error over the cases matching full BB constellations. However, mean translation and mean LCE angle error were less effected by unmatched ilium BBs. When only 3 fragment BBs were matched, the mean LCE angle error was 0.6° larger than the mean LCE angle error associated with all fragment BBs matched. Therefore, the number of matched BBs in each constellation may be used to convey confidences in the estimated poses. When less than 4 fragment BBs are matched, confidence in any rotation and LCE angle would be lowered. For cases when all 4 fragment BBs were matched, but less than 4 ilium BBs were matched, confidence in LCE angle would remain unaffected, however confidence in general rotation would be reduced.

TABLE 4
A summary of the number of BBs detected in each image and the
number matched by the pose estimation process. The total number
of 2D BB detections includes false alarms on screws and BB detections
on the contralateral side. The number of ilium and fragment
BB detections, indicate the number of BBs detected from the
appropriate constellation; a number less than 4 implies missed-
detections. The number of ilium and fragment BB matches is the
number of final correspondences established per constellation
for a given set of ilium and fragment poses.

| | | | Ilium BBs | | Frag. BBs | |
|---|---|---|---|---|---|---|
| Surgery | Proj. | #Total Det. | # Det. | # Match | # Det. | # Match |
| 1 Left | 1 | 13 | 4 | 2 | 4 | 4 |
| 1 Left | 2 | 12 | 4 | 4 | 4 | 4 |
| 1 Left | 3 | 21 | 3 | 2 | 4 | 4 |
| 1 Right | 1 | 11 | 4 | 4 | 4 | 4 |
| 1 Right | 2 | 9 | 4 | 2 | 4 | 4 |
| 1 Right | 3 | 10 | 4 | 4 | 4 | 4 |
| 2 Left | 1 | 7 | 3 | 3 | 4 | 4 |
| 2 Left | 2 | 11 | 3 | 3 | 4 | 4 |
| 2 Left | 3 | 14 | 3 | 3 | 4 | 4 |
| 2 Right | 1 | 7 | 3 | 3 | 4 | 4 |
| 2 Right | 2 | 10 | 3 | 3 | 4 | 4 |
| 2 Right | 3 | 13 | 4 | 3 | 4 | 4 |
| 3 Left | 1 | 10 | 4 | 4 | 4 | 4 |
| 3 Left | 2 | 15 | 4 | 4 | 3 | 3 |
| 3 Left | 3 | 8 | 4 | 4 | 4 | 4 |
| 3 Right | 1 | 8 | 4 | 3 | 4 | 4 |
| 3 Right | 2 | 7 | 3 | 3 | 4 | 4 |
| 3 Right | 3 | 9 | 4 | 3 | 3 | 3 |

Highlighting the robustness of the method, all LCE errors remained below the 3° error threshold, even when BBs were missing from the view or occluded. The method was also robust against BBs which were displaced from the pelvis, but remained in the field of view and detected. This was demonstrated on the right side of specimen 3, where an ilium BB had been dislodged from the bone and was detected in views 1 and 2. Since the P3P solver does not return solutions for non-rigid transformations of the constellations, the displaced BB was not matched, despite the detection of all four ilium BBs in these views.

Average BB reconstruction errors for specimen 1 were greater than those of specimens 2 and 3. This was most likely caused by larger 2D BB localization errors for specimen 1. This is to be expected, since the BBs used for specimen 1 were larger than those used for specimens 2 and 3. Only one of the six LCE errors for specimen 1 was greater than 1°, indicating that the proposed method is not dependent on a single size of BBs.

The performance of the method compares favorably to the fiducial-free method (FFM) proposed in [17]. When a postoperatively segmented fragment shape was retrospectively used for pose estimation, the FFM was reported to have mean rotation error of 2.2°, mean translation error of 2.2 mm, and mean LCE error of 1.1°. The mean rotation error of the proposed method is only slightly larger than the FFM mean rotation error, while translation and LCE angle errors of the proposed method are slightly smaller. When the FFM uses an intraoperatively refined version of a preoperatively planned fragment shape, the mean rotation, translation, and LCE angle errors increase to 3.5°, 2.5 mm, and 1.8°, respectively. Considering that an accurate segmentation of the fragment shape is not available intraoperatively, the method proposed in this paper provides a more accurate assessment of fragment pose and femoral head coverage than the FFM.

In the current state of practice, preoperative CT data cannot be effectively used for intraoperative assessment of anatomical angles. As a result, contemporary preoperative imaging usually consists solely of standing radiographs. Although the proposed method requires a preoperative CT of the patient to be collected, the patient specific CT may be replaced with a statistical atlas of pelvis anatomy [56] in the future. In this approach, the patient's anatomy would be reconstructed using a deformable 2D/3D registration between patient-specific 2D X-ray images and the atlas [57-60]. A precise cartilage model is required for a comprehensive biomechanical analysis, including estimates of the joint contact pressure [6]. Since a statistical atlas may not be capable of satisfactorily reconstructing the cartilage model, a lower-dose, partial CT of the patient's acetabulum may be used to augment the statistical model [61, 62].

The only manual portion of the intraoperative pipeline is the annotation of anatomical landmarks during BB reconstruction. Using recent advances in fluoroscopic deep learning [63], we believe it may be possible for these landmarks to be localized automatically. This would result in a completely automatic intraoperative pipeline, further reducing the impact on existing surgical workflows.

Although the registration framework leveraged from [17] is a highly optimized C++library with OpenCL GPU acceleration, the pruning algorithms described in this paper were implemented as serial C++routines. We believe faster execution times should be possible, since the candidate poses and correspondences evaluated in each pruning phase are not dependent on one another, and the computations may be done in parallel.

Due to the possibility of BBs becoming dislodged from the bone, some clinicians may find the permanent insertion of BBs into patients unacceptable. We believe that this outcome may be avoided by using structures temporarily affixed to the bones during surgery, and then removed after the fragment is satisfactorily fixed in place. The development of such structures is the topic of future work, however two possibilities include: a deformable mesh of BBs that could be impressed on the bone surface, or percutaneously inserted wires which would pierce into the pelvis bone.

The method may also be used for cadaveric PAO training. Pose estimates provided by the system could act as feedback for the mental estimates of the surgeon. In this way, the system may improve surgeons' association of tactile sensing and fluoroscopic interpretation with a fragment's true pose.

Future work will also apply the techniques developed here for fluoroscopic osteotome tracking during PAO. [17] The osteotome could be augmented with a pattern of BBs about it's shaft to enable processing similar to what is done on the acetabular fragment here.

### Conclusion

This example according to an embodiment of the current invention provides a new method for pose estimation of acetabular fragments using flouroscopy and two constellations of intraoperatively implanted BBs. Cadaveric studies have shown that the method is able to provide clinically accurate estimates of the LCE angle, a well-established indicator of femoral head coverage. Once the BB constellations have been reconstructed in 3D, all fragment poses are calculated automatically using a single-view, and in sub-second runtime. No other surgical equipment beyond a flat panel C-Arm and BB injector is required. The C-Arm does not need to be calibrated, encoded, or motorized. Unlike other fluoroscopic approaches, accurate knowledge of the bone fragment's shape is not necessary. For these reasons, the proposed method provides minimal deviation from the standard surgical workflow, and should be easily mastered by clinicians already performing RSA.

### Appendix A: Intensity-Based 2D/3D Registration Parameters

The pelvis-as-fiducial, intensity-based, registration parameters described in [17] are exactly those used for the pre-osteotomy BB reconstruction phase. Each registration in the reconstruction phase runs two resolutions, 8× and 4×downsampling in 2D. In order to overcome large initialization offsets from ground truth, a computationally expensive, evolutionary optimization strategy is used at the 8×level. The less computation-intensive, BOBYQA strategy [64], is used for optimization at the second level.

In order to avoid delays in the surgical workflow, a small execution time is desired during the post-osteotomy pose estimation phase. Therefore, the BOBYQA strategy is used at a single resolution level of 8×downsampling in 2D. We believe a local optimization strategy is sufficient, since solutions reported by the P3P solver, using correct BB correspondences, should lie within some convex ball of the ground truth pose. Box constraints on the se(3) parameter space are specified as in (2), where the X and Y axes are roughly aligned with image columns and rows, respectively, and the Z axis is aligned with the source-to-detector axis.

{±15°,±15°,±30°,±50,°±50°,±100°} (2)

All other registration parameters remain identical to those of the reconstruction phase.

### Appendix B: Pre-Osteotomy BB Reconstruction

Denote the sets of 2D detected BB locations as Pᵥ⊂ [symbol] ² for each view v=1, 2, 3. Nᵥ=|Pᵥ| indicates the number of BBs detected in each view. Let τ: [symbol] ( [symbol] ²)→ [symbol] ³ denote the triangulation operator used to reconstruct a 3D point from a collection of 2D points; [symbol] indicates the power set operator. Let D: [symbol] ³→ [symbol] denote the minimum distance of between a 3D point and the pelvis surface. Let [symbol] ᵥ: [symbol] ³→ [symbol] ² denote the projection operator, applying a perspective projection of 3D points into the imaging plane of view v. As shown in (3), an initial set of correspondences and 3D triangulations, A, are computed for each of the candidate correspondences and any points lying further than T mm away from the pelvis surface are pruned.

A={(p,q,τ(p,q))|p∈P₁,q∈P₂,D(τ(p,q))<T} (3)

The remaining triangulated points are re-projected into the third view and the 2D distances to each BB detection are recorded, shown in (4).

B={(p,q,r,d)|(p,q,x)∈A,r∈P₃,d=∥ [symbol] (x)−r∥₂} (4)

The following book-keeping sets are initialized: the final set of 3D reconstructed points C={ }, and sets indicating whether a 2D BB detection has been used for 3D reconstruction, Rᵥ={ } for v=1, 2, 3. We now iterate through B in increasing order according to the re-projection component, d. For each (p, q, r, d)∈B, if p∉R₁, q∉R₂, and r∉R₃, let y=τ(p, q, r). If [symbol] (y)<T, then the point is a suitable reconstruction; update the bookkeeping sets: C=C∪{y}, R₁=R₁∪{p}, R₂=R₂∪{q}, and R₃=R₃∪{r}. Once iteration over B is complete C represents the final set of 3D BB reconstructions. Iteration may be terminated early when any Rᵥ is equal to Pᵥ for v=1, 2, 3.

### Appendix C: Obtaining P3P Solutions

We wish to find plausible transformations that rigidly map three 3D model points into a C-Arm coordinate frame, so that their projected locations in 2D match a set of corresponding 2D points. Let B₁, B₂, B₃ denote the 3D model points and let b₁, b₂, b₃ be their (hypothesized) corresponding 2D points in the fluoroscopic image. The problem is simplified by assuming that the approximate depth, or proportion along the source-to-detector line, of B₁ in the C-Arm frame is known. Given this information, we know the location of B₁ with respect to the C-Arm, denoted as \tilde{B}₁. For j=2, 3, let \tilde{B}j(t)=s+t(\hat{b}ⱼ−s) denote the lines which B₂ and B₃, with respect to the C-Arm, may possibly lie on. The X-ray source position is denoted by s and the 3D location on the X-ray detector, corresponding to bⱼ, is denoted by \hat{b}ⱼ. For specific values of t₂ and t₃, a potential pose is given by solving the 3D/3D corresponding point set registration [65] between {\tilde{B}₁, \tilde{B}₂(t₂), \tilde{B}₃(t₃)} and {B₁, B₂, B₃}. We find the four possible combinations of t2 and t3 and use the known shape of the 3D model to prune implausible poses.

Let lᵢⱼ=∥Bᵢ−Bⱼ∥₂ denote an inter-BB distance of the 3D model; the lᵢⱼ are known quantities. Let l̅ᵢⱼ=∥\tilde{B}₁−\tilde{B}ⱼ(t)∥₂; the \tilde{l}₁ⱼ(t) are unknown quantities. Using l₁₂ and l₁₃, we find plausible values of t for B̅₂ and B̅₃ by solving (5) for j=2, 3.

minₜ(l₁ⱼ²−\hat{l}₁ⱼ²(t))² (5)

Using MATLAB 2019a, derivatives and formulas for the possible minimizers of (5) were symbolically calculated; a maximum of 2 minimizers are possible. Let tⱼ⁽¹⁾ and tⱼ⁽²⁾ denote the two solutions of (5) for j=2, 3. Poses are pruned when (6), (7) and (8) are not satisfied for the combinations of j=2, 3, k=1, 2, and m=1, 2.

$$0.6 \leq {t}_{j}^{( k )} \leq 1. \qquad ( 6 )$$

$$1 - ε \leq \frac{{\hat{l}}_{1 j} ( {t}_{j}^{( k )} )}{{l}_{1 j}} \leq 1 + ε \qquad ( 7 )$$

$$1 - ε \leq \frac{{\vert {\hat{B}}_{2} ( {t}_{2}^{( k )} ) - {\hat{B}}_{3} ( {t}_{3}^{( m )} ) ‖}_{2}}{{l}_{23}} \leq 1 + ε \qquad ( 8 )$$

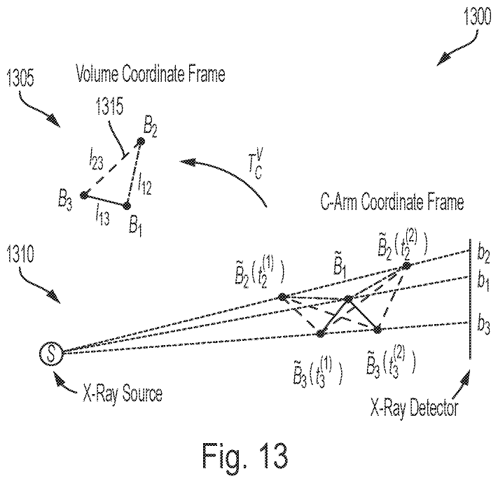

Pruning using (6) constrains objects to lie closer to the X-ray detector than the X-ray source. Pruning using (7) and (8) {\tilde{B}₁, \tilde{B}₂(t₂), \tilde{B}₃(t₃)} to have the same shape as {B₁, B₂, B₃}. The pruning should be conducted in a greedy fashion in order to avoid unnecessary computation. A toy example depicting the geometries described here is shown in FIG. 13. For all experiments in this paper, ϵ=0.01.

FIG. 13 illustrates a toy example 1300 of the P3P problem showing the four possible solutions when mapping the BB constellation 1305 into the C-Arm coordinate frame 1310. This drawing represents a specific source-to-detector distance used to estimate \tilde{B}₁. For each of B₂ and B₃, two possible locations with respect to the C-Arm are shown. The inter-BB length to B₁ is preserved for all 4 solutions. However, visual comparisons of the dashed purple line 1315 in the volume coordinate frame with the corresponding lines in the C-Arm coordinate frame reveal that none of the candidate lengths between B₂ and B₃ are valid. Therefore, no solutions would be reported for this source-to-detector distance.

Similar to the method of Appendix A.3 in [55], we perform this process over a range of \tilde{B}₁. A minimum of zero, and a maximum of four, poses are identified for each depth.

### Appendix D: Fluoroscopy Used for Pose Estimation

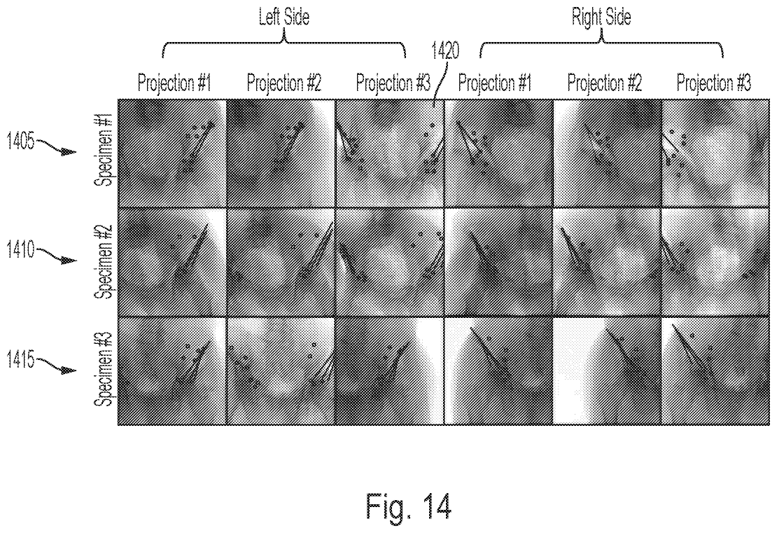

FIG. 14 illustrates fluoroscopic images used for pose estimation in the cadaver surgeries. The detected BBs are overlaid as yellow circles. For specimen 1 1405, the larger radius parameter passed to the radial symmetry method causes several false detections on the screws. Only the smaller injected BBs are detected for the views of specimens 2 1410 and 3 1415; the larger BBs were used for establishing ground truth and not intraoperative pose estimation. Table 4 lists the total number of BB detections in each image. Projection 3 1420 on the left side of specimen 1 shows an example of an excessive number of detections (21), with 8 detections corresponding to BBs on the contralateral side, 6 false alarms triggered by screws, and the remaining 7 detections corresponding to the desired ipsilateral BBs.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. Gala, L., Clohisy, J. C., Beaulé, P. E.: Hip dysplasia in the young adult. J. Bone Joint Surg.-Am Vol. 98(1), 63-73 (2016)
2. Ganz, R., Klaue, K., Vinh, T. S., Mast, J. W.: A new periacetabular osteotomy for the treatment of hip dysplasias technique and preliminary results. Clin. Orthop. Relat. Res. 232, 26-36 (1988)
3. Wiberg, G.: Studies on dysplastic acetabulum and congenital subluxation of the hip joint with special reference to the complications of osteoarthritis. Acta Chir Scand 83(58) (1939)
4. Troelsen, A.: Surgical advances in periacetabular osteotomy for treatment of hip dysplasia in adults. Acta Orthop 80(sup332), 1-33 (2009)
5. Armand, M., Lepisto\ddot{ }, J., Tallroth, K., Elias, J., Chao, E.: Outcome of periacetabular osteotomy: joint contact pressure calculation using standing AP radiographs, 12 patients followed for average 2 years. Acta Orthop 76(3), 303-313 (2005)
6. Armiger, R. S., Armand, M., Tallroth, K., Lepisto\ddot{ }, J., Mears, S. C.: Three-dimensional mechanical evaluation of joint contact pressure in 12 periacetabular osteotomy patients with 10-year follow-up. Acta Orthop 80(2), 155-161 (2009)
7. Hipp, J. A., Sugano, N., Millis, M. B., Murphy, S. B.: Planning acetabular redirection osteotomies based on joint contact pressures. Clin. Orthop. Relat. Res. 364, 134-143 (1999)
8. Niknafs, N., Murphy, R. J., Armiger, R. S., Lepistö, J., Armand, M.: Biomechanical factors in planning of periacetabular osteotomy. Front Bioeng Biotechnol 1 (2013)
9. Liu, L., Ecker, T. M., Schumann, S., Siebenrock, K. A., Zheng, G.: Evaluation of constant thickness cartilage models vs. patient specific cartilage models for an optimized computer-assisted planning of periacetabular osteotomy. PLoS ONE 11(1), e0146452 (2016)
10. Selvik, G.: Roentgen stereophotogrammetric analysis. Acta Radiol 31(2), 113-126 (1990)
11. Akiyama, H., Goto, K., So, K., Nakamura, T.: Computed tomography-based navigation for curved periacetabular osteotomy. J Orthop Sci 15(6), 829-833 (2010)
12. Langlotz, F., Stucki, M., Bächler, R., Scheer, C., Ganz, R., Berlemann, U., Nolte, L. P.: The first twelve cases of computer assisted periacetabular osteotomy. Comput. Aided Surg. 2(6), 317-326 (1997)
13. Liu, L., Zheng, G., Bastian, J. D., Keel, M. J. B., Nolte, L. P., Siebenrock, K. A., Ecker, T. M.: Periacetabular osteotomy through the pararectus approach: technical feasibility and control of fragment mobility by a validated surgical navigation system in a cadaver experiment. Int Orthop 40(7), 1389-1396 (2016)
14. Murphy, R. J., Armiger, R. S., Lepistö, J., Mears, S. C., Taylor, R. H., Armand, M.: Development of a biomechanical guidance system for periacetabular osteotomy. Int J Comput Assist Radiol Surg 10(4), 497-508 (2015)
15. Takao, M., Nishii, T., Sakai, T., Sugano, N.: Comparison of rotational acetabular osteotomy performed with navigation by surgeons with different levels of experience of osteotomies. Int J Comput Assist Radiol Surg 12(5), 841-853 (2017)
16. Troelsen, A., Elmengaard, B., Søballe, K.: A new minimally invasive transsartorial approach for periacetabular osteotomy. J. Bone Joint Surg.-Am Vol. 90(3), 493-498 (2008)
17. Grupp, R. B., Hegeman, R., Murphy, R., Alexander, C., Otake, Y., McArthur, B., Armand, M., Taylor, R. H.: Pose estimation of periacetabular osteotomy fragments with intraoperative X-ray navigation. IEEE Trans. Biomed. Eng. (2019)
18. Langlotz, F., Bächler, R., Berlemann, U., Nolte, L. P., Ganz, R.: Computer assistance for pelvic osteotomies. Clin. Orthop. Relat. Res. 354, 92-102 (1998)
19. Mayman, D. J., Rudan, J., Yach, J., Ellis, R.: The kingston periacetabular osteotomy utilizing computer enhancement: a new technique. Comput Aided Surg 7(3), 179-186 (2002)
20. Otsuki, B., Takemoto, M., Kawanabe, K., Awa, Y., Akiyama, H., Fujibayashi, S., Nakamura, T., Matsuda, S.: Developing a novel custom cutting guide for curved periacetabular osteotomy. Int Orthop 37(6), 1033-1038 (2013)
21. Radermacher, K., Portheine, F., Anton, M., Zimolong, A., Kaspers, G., Rau, G., Staudte, H. W.: Computer assisted orthopaedic surgery with image based individual templates. Clin. Orthop. Relat. Res. 354, 28-38 (1998)
22. De Raedt, S., Mechlenburg, I., Stilling, M., Rømer, L., Murphy, R. J., Armand, M., Lepistö, J., de Bruijne, M., Søballe, K.: Reliability of computer-assisted periacetabular osteotomy using a minimally invasive approach. Int J Comput Assist Radiol Surg 13(12), 2021-2028 (2018)
23. Murphy, R. J., Armiger, R. S., Lepistö, J., Armand, M.: Clinical evaluation of a biomechanical guidance system for periacetabular osteotomy. J Orthop Surg Res 11(1), 1 (2016)
24. Markelj, P., Tomaz ̆eviè, D., Likar, B., Pernus ̆, F.: A review of 3D/2D registration methods for image-guided interventions. Med Image Anal 16(3), 642-661 (2012)
25. Valstar, E. R., Nelissen, R. G., Reiber, J. H., Rozing, P. M.: The use of roentgen stereopho-togrammetry to study micromotion of orthopaedic implants. ISPRS J Photogramm 56(5-6), 376-389 (2002)
26. Kärrholm, J., Hansson, L. I., Selvik, G.: Changes in tibiofibular relationships due to growth disturbances after ankle fractures in children. J. Bone Joint Surg.-Am Vol. 66(8), 1198-1210 (1984)
27. Mechlenburg, I., Kold, S., Rømer, L., Søballe, K.: Safe fixation with two acetabular screws after Ganz periacetabular osteotomy. Acta Orthop 78(3), 344-349 (2007)
28. De Bruin, P., Kaptein, B., Stoel, B., Reiber, J., Rozing, P., Valstar, E.: Image-based RSA: Roentgen stereophotogrammetric analysis based on 2D-3D image registration. J Biomechan 41(1), 155-164 (2008)
29. Seehaus, F., Olender, G. D., Kaptein, B. L., Ostermeier, S., Hurschler, C.: Markerless roentgen stereophotogrammetric analysis for in vivo implant migration measurement using three dimensional surface models to represent bone. J Biomechan 45(8), 1540-1545 (2012)
30. Murphy, R., Otake, Y., Lepistö, J., Armand, M.: Computer-assisted X-ray image-based navigation of periacetabular osteotomy with fiducial based 3D acetabular fragment tracking. In: Orthop. Proc., vol. 95, pp. 84-84. The British Editorial Society of Bone & Joint Surgery (2013)
31. Armand, M., Grupp, R., Murphy, R., Hegman, R., Armiger, R., Taylor, R., McArthur, B., Lepisto, J.: Biomechanical guidance system for periacetabular osteotomy. In: Intelligent Orthopaedics, pp. 169-179. Springer (2018)
32. Choi, J. H., Maier, A., Keil, A., Pal, S., McWalter, E. J., Beaupré, G. S., Gold, G. E., Fahrig, R.: Fiducial marker-based correction for involuntary motion in weight-bearing C-arm CT scanning of knees. II. Experiment. Med Phys 41(6Part1), 061902 (2014)
33. Dang, H., Otake, Y., Schafer, S., Stayman, J. W., Kleinszig, G., Siewerdsen, J.: Robust methods for automatic image-to-world registration in cone-beam CT interventional guidance. Med Phys 39(10), 6484-6498 (2012)
34. Hamming, N., Daly, M., Irish, J., Siewerdsen, J.: Automatic image-to-world registration based on X-ray projections in cone-beam CT-guided interventions. Med Phys 36(5), 1800-1812 (2009)
35. Yaniv, Z.: Localizing spherical fiducials in C-arm based cone-beam CT. Med Phys 36(11), 4957-4966 (2009)
36. Yang, J., Wang, Y., Liu, Y., Tang, S., Chen, W.: Novel approach for 3-d reconstruction of coronary arteries from two uncalibrated angiographic images. IEEE Trans. Image Process. 18(7), 1563-1572 (2009)
37. Blondel, C., Malandain, G., Vaillant, R., Ayache, N.: Reconstruction of coronary arteries from a single rotational X-ray projection sequence. IEEE Trans. Med. Imag. 25(5), 653-663 (2006)
38. Snavely, N., Seitz, S. M., Szeliski, R.: Modeling the world from internet photo collections. Int. J. Comput. Vis. 80(2), 189-210 (2008)
39. Westoby, M. J., Brasington, J., Glasser, N. F., Hambrey, M. J., Reynolds, J.: structure-from-motionphotogrammetry: A low-cost, effective tool for geoscience applications. Geomorphology 179, 300-314 (2012)
40. Schonberger, J. L., Frahm, J. M.: Structure-from-motion revisited. In: Proc. IEEE Conf. Comput. Vis. Pattern Recognit., pp. 4104-4113 (2016)
41. Schweikard, A., Glosser, G., Bodduluri, M., Murphy, M. J., Adler, J. R.: Robotic motion compensation for respiratory movement during radiosurgery. Comput. Aided Surg. 5(4), 263-277 (2000)
42. Litzenberg, D., Dawson, L. A., Sandler, H., Sanda, M. G., McShan, D. L., Ten Haken, R. K., Lam, K. L., Brock, K. K., Balter, J. M.: Daily prostate targeting using implanted radiopaque markers. Int. J. Radiat. Oncol. Biol. Phys. 52(3), 699-703 (2002)
43. Aubry, J. F., Beaulieu, L., Girouard, L. M., Aubin, S., Tremblay, D., Laverdière, J., Vigneault, E.: Measurements of intrafraction motion and interfraction and intrafraction rotation of prostate by three-dimensional analysis of daily portal imaging with radiopaque markers. Int. J. Radiat. Oncol. Biol. Phys. 60(1), 30-39 (2004)
44. Yuan, X., Ryd, L., Tanner, K., Lidgren, L.: Roentgen single-plane photogrammetric analysis (RSPA) a new approach to the study of musculoskeletal movement. J. Bone Joint Surg.-Br Vol. 84(6), 908-914 (2002)
45. Tang, T. S., Ellis, R. E., Fichtinger, G.: Fiducial registration from a single x-ray image: a new technique for fluoroscopic guidance and radiotherapy. In: Proc. Med. Image Comput. Comput.-Assist. Interv, pp. 502-511. Springer (2000)
46. Kang, X., Armand, M., Otake, Y., Yau, W. P., Cheung, P. Y., Hu, Y., Taylor, R. H.: Robustness and accuracy of feature-based single image 2-D-3-D registration without correspondences for image-guided intervention. IEEE Trans. Biomed. Eng. 61(1), 149-161 (2013)
47. Jain, A. K., Mustafa, T., Zhou, Y., Burdette, C., Chirikjian, G. S., Fichtinger, G.: Ftraca robust fluoroscope tracking fiducial. Med Phys 32(10), 3185-3198 (2005)
48. Steger, T., Hoßbach, M., Wesarg, S.: Marker detection evaluation by phantom and cadaver experiments for C-arm pose estimation pattern. In: Proc. SPIE, vol. 8671, p. 86711V. International Society for Optics and Photonics (2013)
49. Tang, T. S., MacIntyre, N., Gill, H., Fellows, R., Hill, N., Wilson, D., Ellis, R. E.: Accurate assessment of patellar tracking using fiducial and intensity-based fluoroscopic techniques. Med Image Anal 8(3), 343-351 (2004)
50. Ioppolo, J., Börlin, N., Bragdon, C., Li, M., Price, R., Wood, D., Malchau, H., Nivbrant, B.: Validation of a low-dose hybrid RSA and fluoroscopy technique: Determination of accuracy, bias and precision. J Biomechan 40(3), 686-692 (2007)
51. Krèah, M., Székely, G., Blanc, R.: Fully automatic and fast segmentation of the femur bone from 3D-CT images with no shape prior. In: Proc. IEEE Intl. Symp. Biomed. Imag, pp. 2087-2090 (2011)
52. Nikou, C., Jaramaz, B., DiGioia, A. M., Levison, T. J.: Description of anatomic coordinate systems and rationale for use in an image-guided total hip replacement system. In: Proc. Med. Image Comput. Comput.-Assist. Interv, pp. 1188-1194 (2000)
53. Parthasarathy, R.: Rapid, accurate particle tracking by calculation of radial symmetry centers. Nature Methods 9(7), 724 (2012)
54. Hartley, R., Zisserman, A.: Multiple view geometry in computer vision. Cambridge university press (2003)
55. Fischler, M. A., Bolles, R. C.: Random sample consensus: a paradigm for model fitting with applications to image analysis and automated cartography. Commun. ACM 24(6), 381-395 (1981)
56. Otake, Y., Murphy, R., Grupp, R., Sato, Y., Taylor, R., Armand, M.: Comparison of optimization strategy and similarity metric in atlas-to-subject registration using statistical deformation model. In: Proc. SPIE, pp. 94150Q-94150Q. International Society for Optics and Photonics (2015)
57. Sadowsky, O., Chintalapani, G., Taylor, R.: Deformable 2D-3D registration of the pelvis with a limited field of view, using shape statistics. In: Proc. Med. Image Comput. Comput.-Assist. Interv, pp. 519-526 (2007)
58. Hurvitz, A., Joskowicz, L.: Registration of a CT-like atlas to fluoroscopic X-ray images using intensity correspondences. Int J Comput Assist Radiol Surg 3(6), 493 (2008)
59. Zheng, G.: Statistical shape model-based reconstruction of a scaled, patient-specific surface model of the pelvis from a single standard AP X-ray radiograph. Med Phys 37(4), 1424-1439 (2010)
60. Kang, X., Yau, W. P., Taylor, R. H.: Simultaneous pose estimation and patient-specific model reconstruction from single image using maximum penalized likelihood estimation (MPLE). Pattern Recognit. 57, 61-69 (2016)
61. Chintalapani, G., Murphy, R., Armiger, R. S., Lepisto, J., Otake, Y., Sugano, N., Taylor, R. H., Armand, M.: Statistical atlas based extrapolation of CT data. Proc. SPIE pp. 762539-762539 (2010)
62. Grupp, R., Otake, Y., Murphy, R., Parvizi, J., Armand, M., Taylor, R.: Pelvis surface estimation from partial CT for computer-aided pelvic osteotomies. In: Orthop. Proc., vol. 98, pp. 55-55. The British Editorial Society of Bone & Joint Surgery (2016)
63. Bier, B., Goldmann, F., Zaech, J. N., Fotouhi, J., Hegeman, R., Grupp, R., Armand, M., Osgood, G., Navab, N., Maier, A., et al.: Learning to detect anatomical landmarks of the pelvis in X-rays from arbitrary views. Int J Comput Assist Radiol Surg pp. 1-11
64. Powell, M. J.: The BOBYQA algorithm for bound constrained optimization without derivatives. Cambridge NA Report NA2009/06, University of Cambridge, Cambridge (2009)
65. Horn, B. K.: Closed-form solution of absolute orientation using unit quaternions. J Opt Soc Am A 4(4), 629-642 (1987)

1. A computerized system for representing a relative change in position and/or orientation of a bone section for use during a surgical procedure, said computerized system comprising data processing circuits configured to:
receive preoperative x-ray computed tomography (CT) image data of a bone that will have a portion separated and moved during said surgical procedure;
receive x-ray image data for a plurality of two-dimensional x-ray images, each two-dimensional x-ray image being a different view of said bone during said surgical procedure prior to having said portion separated and moved during said surgical procedure, wherein said bone has a first fiducial marker fixed relative to said portion of said bone that will be separated and moved and a second fiducial marker fixed relative to a portion of said bone that will remain substantially stationary during said surgical procedure, said first and second fiducial markers each having at least three radio-opaque points to be identifiable in said plurality of two-dimensional x-ray images and that remain substantially fixed with respect to each other within each respective first and second fiducial marker during said surgical procedure;
determine a position of said at least three radio-opaque points in each of said first and second fiducial markers relative to a three-dimensional representation of said bone from said preoperative CT image data of said bone;
receive after said portion of said bone is separated and moved during said surgical procedure, a single two-dimensional x-ray image data of at least a portion of said bone that includes both said first and second fiducial markers;
estimate at least one of said relative change in position or said relative change of orientation of said portion of said bone that was separated and moved during said surgical procedure using said single two-dimensional x-ray image data; and
provide information to a user representing at least one of said relative change in position or said relative change of orientation of said portion of said bone that was separated and moved during said surgical procedure based on said estimating.

2. The computerized system according to claim 1, wherein said x-ray image data for said plurality of two-dimensional x-ray images and said single two-dimensional x-ray image data are all fluoroscopy x-ray image data.

3. The computerized system according to claim 1, wherein said estimating, by said computerized system, uses known information to prune out computation that do not have to be performed to thereby speed up computations to be used for said estimating.

4. The computerized system according to claim 3, wherein said known information is at least one of known relative positions of said at least three radio-opaque points of said first fiducial marker, a known position of an x-ray transmitter and receiver being used during said surgical procedure and a position of a patient relative thereto, a position and orientation of said patient during said surgical procedure, a maximum amount of rotation that said portion of said bone that will be separated and moved will encounter during said surgical procedure, or a maximum amount of displacement that said portion of said bone that will be separated and moved will encounter during said surgical procedure.

5. The computerized system according to claim 1, wherein said first and second fiducial markers are each a constellation of at least three BBs.

6. The computerized system according to claim 1, wherein said surgical procedure is a periacetabular osteotomy to treat developmental dysplasia of the hip by reorienting the hip joint for increased femoral head coverage.

7. A method of representing a relative change in position and/or orientation of a bone section by a computer for use during a surgical procedure, comprising:
receiving, by said computer, preoperative x-ray computed tomography (CT) image data of a bone that will have a portion separated and moved during said surgical procedure;
receiving, by said computer, x-ray image data for a plurality of two-dimensional x-ray images, each two-dimensional x-ray image being a different view of said bone during said surgical procedure prior to having said portion separated and moved during said surgical procedure, wherein said bone has a first fiducial marker fixed relative to said portion of said bone that will be separated and moved and a second fiducial marker fixed relative to a portion of said bone that will remain substantially stationary during said surgical procedure, said first and second fiducial markers each having at least three radio-opaque points to be identifiable in said plurality of two-dimensional x-ray images and that remain substantially fixed with respect to each other within each respective first and second fiducial marker during said surgical procedure;
determining, by said computer, a position of said at least three radio-opaque points in each of said first and second fiducial markers relative to a three-dimensional representation of said bone from said preoperative CT image data of said bone;
receiving, by said computer after said portion of said bone is separated and moved during said surgical procedure, a single two-dimensional x-ray image data of at least a portion of said bone that includes both said first and second fiducial markers;
estimating, by said computer, at least one of said relative change in position or said relative change of orientation of said portion of said bone that was separated and moved during said surgical procedure using said single two-dimensional x-ray image data; and
providing, by said computer, information to a user representing at least one of said relative change in position or said relative change of orientation of said portion of said bone that was separated and moved during said surgical procedure based on said estimating.

8. The method according to claim 7, wherein said x-ray image data for said plurality of two-dimensional x-ray images and said single two-dimensional x-ray image data are all fluoroscopy x-ray image data.

9. The method according to claim 7, wherein said estimating, by said computer, uses known information to prune out computation that do not have to be performed to thereby speed up computations to be used for said estimating.

10. The method according to claim 9, wherein said known information is at least one of known relative positions of said at least three radio-opaque points of said first fiducial marker, a known position of an x-ray transmitter and receiver being used during said surgical procedure and a position of a patient relative thereto, a position and orientation of said patient during said surgical procedure, a maximum amount of rotation that said portion of said bone that will be separated and moved will encounter during said surgical procedure, or a maximum amount of displacement that said portion of said bone that will be separated and moved will encounter during said surgical procedure.

11. The method according to claim 7, wherein said first and second fiducial markers are each a constellation of at least three BBs.

12. The method according to claim 7, wherein said surgical procedure is a periacetabular osteotomy to treat developmental dysplasia of the hip by reorienting the hip joint for increased femoral head coverage.

13. A computer-readable medium comprising computer-executable code for representing a relative change in position and/or orientation of a bone section for use during a surgical procedure, which when executed by a computer causes said computer to:
receive preoperative x-ray computed tomography (CT) image data of a bone that will have a portion separated and moved during said surgical procedure;
receive x-ray image data for a plurality of two-dimensional x-ray images, each two-dimensional x-ray image being a different view of said bone during said surgical procedure prior to having said portion separated and moved during said surgical procedure, wherein said bone has a first fiducial marker fixed relative to said portion of said bone that will be separated and moved and a second fiducial marker fixed relative to a portion of said bone that will remain substantially stationary during said surgical procedure, said first and second fiducial markers each having at least three radio-opaque points to be identifiable in said plurality of two-dimensional x-ray images and that remain substantially fixed with respect to each other within each respective first and second fiducial marker during said surgical procedure;
determine a position of said at least three radio-opaque points in each of said first and second fiducial markers relative to a three-dimensional representation of said bone from said preoperative CT image data of said bone;
receive after said portion of said bone is separated and moved during said surgical procedure, a single two-dimensional x-ray image data of at least a portion of said bone that includes both said first and second fiducial markers;
estimate at least one of said relative change in position or said relative change of orientation of said portion of said bone that was separated and moved during said surgical procedure using said single two-dimensional x-ray image data; and
provide information to a user representing at least one of said relative change in position or said relative change of orientation of said portion of said bone that was separated and moved during said surgical procedure based on said estimating.

14. The computer-readable medium according to claim 13, wherein said x-ray image data for said plurality of two-dimensional x-ray images and said single two-dimensional x-ray image data are all fluoroscopy x-ray image data.

15. The computer-readable medium according to claim 13, wherein said estimating, by said computer, uses known information to prune out computation that do not have to be performed to thereby speed up computations to be used for said estimating.

16. The computer-readable medium according to claim 15, wherein said known information is at least one of known relative positions of said at least three radio-opaque points of said first fiducial marker, a known position of an x-ray transmitter and receiver being used during said surgical procedure and a position of a patient relative thereto, a position and orientation of said patient during said surgical procedure, a maximum amount of rotation that said portion of said bone that will be separated and moved will encounter during said surgical procedure, or a maximum amount of displacement that said portion of said bone that will be separated and moved will encounter during said surgical procedure.

17. The computer-readable medium according to claim 13, wherein said first and second fiducial markers are each a constellation of at least three BBs.

18. The computer-readable medium according to claim 13, wherein said surgical procedure is a periacetabular osteotomy to treat developmental dysplasia of the hip by reorienting the hip joint for increased femoral head coverage.
